(19) United States
(12) Patent Application Publication
(10) Pub. No.: US 2020/0354706 A1
(43) Pub. Date: Nov. 12, 2020

(54) NOVEL RECOMBINANT BOTULINUM TOXIN WITH INCREASED DURATION OF EFFECT

(71) Applicant: MERZ PHARMA GMBH & CO. KGAA, Frankfurt am Main (DE)

(72) Inventors: Jürgen FREVERT, Berlin (DE); Fred HOFMANN, Potsdam (DE); Marcel JURK, Berlin (DE); Manuela LÓPEZ DE LA PAZ, Liederbach am Taunus (DE); Daniel SCHEPS, Potsdam (DE)

(73) Assignee: MERZ PHARMA GMBH & CO. KGAA, Frankfurt am Main (DE)

(21) Appl. No.: 16/760,377

(22) PCT Filed: Nov. 22, 2017
(86) PCT No.: PCT/EP2017/080117

§ 371 Date: Apr. 29, 2020

(51) Int. Cl. C12N 9/52; A61K 8/66; A61Q 19/08

(52) U.S. Cl. CPC C12N 9/52; A61K 8/66; A61K 38/00; A61Q 19/08; C12Y 304/24069

(57) ABSTRACT

This invention relates to novel recombinant clostridial neurotoxins exhibiting an increased duration of effect and to methods for the manufacture of such recombinant clostridial neurotoxins. These novel recombinant clostridial neurotoxins comprise at least one domain wherein said domain comprises an amino acid sequence consisting of at least 50 amino acid residues, wherein said amino acid sequence consists of at least one proline and at least one alanine residue. The invention relates also to novel recombinant clostridial neurotoxins comprising at least one domain wherein said domain comprises an amino acid sequence consisting of at least 50 amino acid residues, wherein said domain comprises a plurality of specific amino acid repeats consisting of proline, alanine and tyrosine residues, or proline, alanine and glutamine residues, or proline, alanine and threonine residues.

### FIELD OF THE INVENTION

This invention relates to novel recombinant clostridial neurotoxins exhibiting increased duration of effect and to methods for the manufacture of such recombinant clostridial neurotoxins. These novel recombinant clostridial neurotoxins comprise at least one domain wherein said domain comprises an amino acid sequence consisting of at least 50 amino acid residues, wherein said amino acid sequence consists of at least one proline and at least one alanine residue. The invention further relates to novel recombinant clostridial neurotoxins comprising at least one domain wherein said domain comprises an amino acid sequence consisting of at least 50 amino acid residues, wherein said domain comprises a plurality of specific amino acid repeats consisting of proline, alanine and tyrosine residues, or proline, alanine and glutamine residues, or proline, alanine and threonine residues.

### BACKGROUND OF THE INVENTION

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TxNT) function via a similar initial physiological mechanism of action, inhibiting neurotransmitter release from the axon of the affected neuron into the synapse, they differ in their clinical response. While the botulinum toxin acts at the neuromuscular junction and other cholinergic synapses in the peripheral nervous system, inhibiting the release of the neurotransmitter acetylcholine and thereby causing flaccid paralysis, the tetanus toxin acts mainly in the central nervous system, preventing the release of the inhibitory neurotransmitters GABA (gamma-aminobutyric acid) and glycine by degrading the protein synaptobrevin. The consequent overactivity in the muscles results in generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic types, termed BoNT/A through BoNT/G. Most Clostridium botulinum strains produce one type of neurotoxin, but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial endoprotease to obtain the biologically active toxin.

The molecular mechanism of intoxication by TeNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave one or two of the so called SNARE-proteins, which are essential for different steps of neurotransmitter release into the synaptic cleft, e.g. recognition, docking and fusion of neurotransmitter-containing vesicles with the plasma membrane. TeNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

Clostridial neurotoxins display variable durations of action that are serotype specific. The clinical therapeutic effect of BoNT/A lasts approximately 3 months for neuromuscular disorders and 6 to 12 months for hyperhidrosis. The effect of BoNT/E, on the other hand, lasts less than 4 weeks. The longer lasting therapeutic effect of BoNT/A makes it preferable for certain clinical use compared to the other serotypes, for example serotypes B, C₁, D, E, F, G. One possible explanation for the divergent durations of action might be the distinct subcellular localizations of BoNT serotypes. The protease domain of BoNT/A light chain localizes in a punctate manner to the plasma membrane of neuronal cells, co-localizing with its substrate SNAP-25. In contrast, the short-duration BoNT/E serotype LC is cytoplasmic. Membrane association might protect BoNT/A from cytosolic degradation mechanisms allowing for prolonged persistence of BoNT/A in the neuronal cell.

The onset of the paralytic effect is also different in the neurotoxin serotypes. Whereas the onset of effect of BoNT/E in humans is observed after 0.5-1 day, the onset of the effect of BoNT/A in humans is only after 2-3 days. The peak effect is reached in humans after 1-2 days or 3-7 day after injections of BoNT/E or BoNT/A, respectively. Consequently BoNT/A has a late onset and a long duration of the paralytic effect, in contrast the paralytic effect of BoNT/E starts markedly earlier but lasts markedly less long. The reason for this different onset and different duration of effect is not known.

In Clostridium botulinum, the botulinum toxin is formed as a protein complex comprising the neurotoxic component and non-toxic proteins. The accessory proteins embed the neurotoxic component thereby protecting it from degradation by digestive enzymes in the gastrointestinal tract. Thus, botulinum neurotoxins of most serotypes are orally toxic. Complexes with, for example, 450 kDa or with 900 kDa are obtainable from cultures of Clostridium botulinum.

In recent years, botulinum neurotoxins have been used as therapeutic agents in the treatment of dystonias and spasms. Preparations comprising botulinum toxin complexes are commercially available, e.g. from Ipsen Ltd (Dysport®) or Allergan Inc. (Botox®). A high purity neurotoxic component, free of any complexing proteins, is for example available from Merz Pharmaceuticals GmbH, Frankfurt (Xeomin®).

Clostridial neurotoxins are usually injected into the affected muscle tissue, bringing the agent close to the neuromuscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity. Immunologic tolerance to botulinum toxin has been shown to correlate with cumulative doses and in addition on short injection intervals.

At present, clostridial neurotoxins are still predominantly produced by fermentation processes using appropriate Clostridium strains. However, industrial production of clostridial neurotoxin from anaerobic Clostridium culturing is a cumbersome and time-consuming process. Due to the high toxicity of the final product, the procedure must be performed under strict containment. During the fermentation process, the single-chain precursors are proteolytically cleaved by an unknown clostridial protease to obtain the biologically active di-chain clostridial neurotoxin. The degree of neurotoxin activation by proteolytic cleavage varies between different strains and neurotoxin serotypes, which is a major consideration for the manufacture due to the requirement of neurotoxin preparations with a well-defined biological activity. Furthermore, during fermentation processes using Clostridium strains the clostridial neurotoxins are produced as protein complexes, in which the neurotoxic component is embedded by accessory proteins. These accessory proteins have no beneficial effect on biological activity or onset of effect or other pharmacological properties. They can however trigger an immune reaction in the patient, resulting in immunity against the clostridial neurotoxin. Manufacture of recombinant clostridial neurotoxins, which are not embedded by auxiliary proteins, might therefore be advantageous.

Methods for the recombinant expression of clostridial neurotoxins in E. coli are well known in the art (see, for example, WO 00/12728, WO 01/14570, or WO 2006/076902). Furthermore, clostridial neurotoxins have been expressed in eukaryotic expression systems, such as in Pichia pastoris, Pichia methanolica, Saccharomyces cerevisiae, insect cells and mammalian cells (see WO 2006/017749).

Recombinant clostridial neurotoxins may be expressed as single-chain precursors, which subsequently have to be proteolytically cleaved to obtain the final biologically active clostridial neurotoxin. Thus, clostridial neurotoxins may be expressed in high yield in rapidly-growing bacteria as relatively non-toxic single-chain polypeptides.

Furthermore, it might be advantageous to modify clostridial neurotoxin characteristics regarding biological activity, cell specificity, antigenic potential and duration of effect by genetic engineering to obtain recombinant neurotoxins with new therapeutic properties in specific clinical areas. Genetic modification of clostridial neurotoxins might allow altering the mode of action or expanding the range of therapeutic targets.

WO 96/39166 discloses analogues of botulinum toxin comprising amino acid residues which are more resistant to degradation in neuromuscular tissue.

Patent family based on WO 02/08268 (including family member U.S. Pat. No. 6,903,187) discloses a clostridial neurotoxin comprising a structural modification selected from addition or deletion of a leucine-based motif, which alters the biological persistence of the neurotoxin (see also: Fernandez-Salas et al., Proc. Natl. Acad. Sci. U.S.A. 101 (2004) 3208-3213; Wang et al., J. Biol. Chem. 286 (2011) 6375-6385). Fernandez-Salas et al. initially hypothesized that the increased persistence was due to the membrane-binding properties of the dileucine motif (see Fernandez-Salas et al., loc. cit., p. 3211 and 3213). Wang et al. mention this membrane theory (see Wang et al., loc. cit., p. 6376, left column, last full paragraph, and p. 6383, first full paragraph of “Discussion”), but favor an alternative theory: the protection from degradation by proteolysis (see Wang et al., loc. cit., p. 6384, left column, lines 27ff).

WO 2015/132004 describes clostridial neurotoxins comprising a random coil domain, particularly wherein said random coil domain consists of alanine, serine and proline residues, and exhibiting an altered biological persistence.

A botulinum toxin variant exhibiting an increased duration of effect in neuromuscular tissue than naturally occurring botulinum toxins would be advantageous in order to reduce administration frequency and the incidence of neutralizing antibody generation since immunologic tolerance to botulinum toxin is correlated with cumulative doses.

Furthermore, BoNT serotypes naturally exhibiting a short duration of action could potentially be effectively used in clinical applications, if their biological persistence could be enhanced. Modified BoNT/E with an increased duration of action could potentially be used in patients exhibiting an immune reaction against BoNT/A. Moreover, BoNT/E was shown to induce a more severe block of pain mediator release from sensory neurons than BoNT/A. In clinical applications where BoNT/A provides only partial pain relief or in just a subset of patients, such as in the treatment of headaches, or where BoNT/E has been found to be more effective than BoNT/A but gives only short-term therapy, such as in the treatment of epilepsy, BoNT/E with an increased duration of effect might prove useful.

There is a strong demand to produce clostridial neurotoxins with an increased duration of effect in order to allow for reduction of administration frequency and exploitation of the therapeutic potential of BoNT serotypes, which have so far been considered impractical for clinical application due to the short half-life of the respective clinically relevant effect. Ideally, the duration of effect of a particular clostridial neurotoxin could be adjusted in a tailor-made fashion in order to address any particular features and demands of a given indication, such as the amount of neurotoxin being administered, frequency of administration etc. To date, despite the progress that has already been made (see, in particular, WO 2015/132004), such aspects have not been solved satisfactorily.

### OBJECTS OF THE INVENTION

It was an object of the invention to provide alternative recombinant clostridial neurotoxins exhibiting an increased duration of effect, and to establish a reliable and accurate method for manufacturing and obtaining such recombinant clostridial neurotoxins. Such a method and novel precursor clostridial neurotoxins used in such methods would serve to satisfy the great need for recombinant clostridial neurotoxins exhibiting an increased duration of effect.

### SUMMARY OF THE INVENTION

The naturally occurring botulinum toxin serotypes display highly divergent durations of effect, probably due to their distinct subcellular localization. BoNT/A exhibiting the longest persistence was shown to localize in the vicinity of the plasma membrane of neuronal cells, whereas the short-duration BoNT/E serotype is cytosolic. However, additional factors such as degradation, diffusion, and/or translocation rates might have a decisive impact on the differences in the duration of effect for the individual botulinum toxin serotypes.

So far, except for the approach described and claimed in WO 2015/132004, no generally applicable method for modifying clostridial neurotoxins in order to increase their duration of effect is available. Surprisingly, it has been found that alternative recombinant clostridial neurotoxins exhibiting an increased duration of effect can be obtained by cloning a specific sequence encoding a certain domain into a gene encoding a parental clostridial neurotoxin, and by subsequent heterologous expression of the generated construct in recombinant host cells.

Thus, in one aspect, the present invention relates to a recombinant clostridial neurotoxin comprising at least one domain wherein said domain comprises an amino acid sequence consisting of at least 50 amino acid residues, wherein said amino acid sequence consists of at least one proline and at least one alanine residue, or wherein said domain comprises a plurality of amino acid repeats, wherein said repeats consist of proline and alanine residues and wherein no more than six consecutive amino acid residues are identical. The amino acid sequences according to the invention do not include any serine residues.

In another aspect, the present invention relates to recombinant clostridial neurotoxin comprising at least one domain wherein said domain comprises an amino acid sequence consisting of at least 50 amino acid residues, wherein said domain comprises a plurality of amino acid repeats, wherein said repeats consist of an amino acid sequence selected from the group consisting of AYPAAPAPAYPAAPAPYAPA (SEQ ID NO: 1), AQPAAPAPAQPAAPAPQAPA (SEQ ID NO: 2) and ATPAAPAPATPAAPAPTAPA (SEQ ID NO: 3). The amino acid repeats according to the invention do not include any serine residues.

In another aspect, the present invention relates to a composition, in particular to a pharmaceutical composition, comprising the recombinant clostridial neurotoxin of the present invention.

In yet another aspect, the present invention relates to the use of the composition of the present invention for cosmetic treatment.

In yet another aspect, the present invention relates to a method for treating a patient comprising the step of administering a composition comprising the recombinant clostridial neurotoxin of the present invention.

In another aspect, the present invention relates to a method for the generation of the recombinant clostridial neurotoxin of the present invention, comprising the step of obtaining a recombinant nucleic acid sequence encoding a recombinant single-chain precursor clostridial neurotoxin by the insertion of one or more nucleic acid sequences, each encoding said domain, at one or two positions into a nucleic acid sequence encoding a parental clostridial neurotoxin and expressing the protein in a host cell.

In another aspect, the present invention relates to a recombinant single-chain precursor clostridial neurotoxin comprising one or two domains according to the invention.

In another aspect, the present invention relates to a nucleic acid sequence encoding the recombinant single-chain precursor clostridial neurotoxin of the present invention.

In another aspect, the present invention relates to a method for obtaining the nucleic acid sequence of the present invention, comprising the step of inserting one or more nucleic acid sequences encoding said domain at one or two positions into a nucleic acid sequence encoding a parental clostridial neurotoxin.

In another aspect, the present invention relates to a vector comprising the nucleic acid sequence of the present invention, or the nucleic acid sequence obtainable by the method of the present invention.

In another aspect, the present invention relates to a recombinant host cell comprising the nucleic acid sequence of the present invention, the nucleic acid sequence obtainable by the method of the present invention, or the vector of the present invention.

In another aspect, the present invention relates to a method for producing the recombinant single-chain precursor clostridial neurotoxin of the present invention, comprising the step of expressing the nucleic acid sequence of the present invention, or the nucleic acid sequence obtainable by the method of the present invention, or the vector of the present invention in a recombinant host cell, or cultivating the recombinant host cell of the present invention under conditions that result in the expression of said nucleic acid sequence.

### FIGURES

FIG. 1: Schematic presentation of a PA-botulinum toxin A (PA100-rBoNT/A-PA100).

FIG. 2: SDS⋅PAGE of purified PA-botulinum toxin A (PA100-rBoNT/A-PA100). Prior to applying the samples to the gel, ß-mercaptoethanol was added. Lane 1: molecular weight marker. Lane “v.A.” (before activation): purified, non-activated single-chain PA100-rBoNT/A-PA100. Lanes “n.A.” (after activation) and “n.R.” (after purification) show light chain (PA100-LC) and heavy chain (PA100-HC) obtained after activation by thrombin under reducing conditions.

FIG. 3: Mouse running assay with PA100-rBoNT/A-PA100:

Two different dosages of MaJ008 (PA100-rBoNT/A-PA100), i.e. 5 and 6 pg were injected into the M. gastrocnemius of eight mice (volume 20 μl).
A standard of Xeomin (Std. 81208; 0.6 U) and a mean of standard (17 assays) of Xeomin® 81208 (0.6 U) were injected into the M. gastrocnemius of eight mice (volume 20 μl).
DaSch021 (PAS100-rBoNT/A-PAS100) (9 pg) mean value of three assays.

FIG. 4: Schematic presentation of a PAY-botulinum toxin A (PAY100-rBoNT/A-PAY100).

FIG. 5: SDS⋅PAGE of purified PAY-botulinum toxin A (PAY100-rBoNT/A-PAY100). Prior to applying the samples to the gel, ß-mercaptoethanol was added. Lane “M”: molecular weight marker. Lane “v.A.” (before activation): purified, non-activated single-chain PAY100-rBoNT/A-PAY100. Lanes “n.A.” (after activation) and “n.R.” (after purification) show light chain (PAY100-LC) and heavy chain (PAY100-HC) obtained after activation by thrombin under reducing conditions.

FIG. 6: Mouse running assay with PAY100-rBoNT/A-PAY100:

Two different dosages of Dasch084 (PAY100-rBoNT/A-PAY100), i.e. 2 and 4 pg were injected into the M. gastrocnemius of eight mice (volume 20 μl).
A mean of standard (17 assays) of Xeomin® 81208 (0.6 U) was injected into the M. gastrocnemius of eight mice (volume 20 μl).
DaSch021 (PAS100-rBoNT/A-PAS100) (9 pg) mean value of three assays.

FIG. 7: Schematic presentation of a PAQ-botulinum toxin A (PAQ100-rBoNT/A-PAQ100).

FIG. 8: SDS⋅PAGE of purified PAQ-botulinum toxin A (PAQ100-rBoNT/A-PAQ100). Prior to applying the samples to the gel, ß-mercaptoethanol was added. Lane “M”: molecular weight marker. Lane “v.A.” (before activation): purified, non-activated single-chain PAQ100-rBoNT/A-PAQ100. Lanes “n.A.” (after activation) and “n.R.” (after purification) show light chain (PAQ100-LC) and heavy chain (PAQ100-HC) obtained after activation by thrombin under reducing conditions.

FIG. 9: Mouse running assay with PAQ100-rBoNT/A-PAQ100:

Two different dosages of Dasch084 (PAQ100-rBoNT/A-PAQ100), i.e. 6 and 9 pg were injected into the M. gastrocnemius of eight mice (volume 20 μl).
A mean of standard (17 assays) from Xeomin® 81208 (0.6 U) was injected into the M. gastrocnemius of eight mice (volume 20 μl).
DaSch021 (PAS100-rBoNT/A-PAS100) (9 pg) mean value of three assays.

FIG. 10: Schematic presentation of a PAT-botulinum toxin A (PAT100-rBoNT/A-PAT100).

FIG. 11: SDS⋅PAGE of purified PAT-botulinum toxin A (PAT100-rBoNT/A-PAT100). Prior to applying the samples to the gel, ß-mercaptoethanol was added. Lane “M”: molecular weight marker. Lane “v.A.” (before activation): purified, non-activated single-chain PAT100-rBoNT/A-PAT100. Lanes “n.A.” (after activation) and “n.R.” (after purification) show light chain (PAT100-LC) and heavy chain (PAT100-HC) obtained after activation by thrombin under reducing conditions.

FIG. 12: Mouse running assay with PAT100-rBoNT/A-PAT100:

A dosage of Dasch085 (PAT100-rBoNT/A-PAT100), i.e. 11 pg were injected into the M. gastrocnemius of eight mice (volume 20 μl). A mean of standard (17 assays) from Xeomin® 81208 (0.6 U) was injected into the M. gastrocnemius of eight mice (volume 20 μl). DaSch021 (PAS100-rBoNT/A-PAS100) (9 pg) mean value of three assays.

### DETAILED DESCRIPTION OF THE INVENTION

The present invention may be understood more readily by reference to the following detailed description of the invention and the examples included therein.

In one aspect, the present invention relates to a recombinant clostridial neurotoxin comprising at least one domain wherein said domain comprises an amino acid sequence consisting of at least 50 amino acid residues, wherein said amino acid sequence consists of at least one proline and at least one alanine residue, or wherein said domain comprises a plurality of amino acid repeats, wherein said repeats consist of proline and alanine residues and wherein no more than six consecutive amino acid residues are identical. The amino acid sequences according to the invention do not include any serine residues.

In particular embodiments, said recombinant clostridial neurotoxin comprises said domain comprising a plurality of amino acid repeats consisting of (AAPAA PAPAA PAAPA PAAPA)ₙ, with n being an integer selected from 3 to 25, in particular wherein n is 5.

In a further aspect, the present invention relates to a recombinant clostridial neurotoxin comprising at least one domain wherein said domain comprises an amino acid sequence consisting of at least 50 amino acid residues, wherein said domain comprises a plurality of amino acid repeats, wherein said repeats consist of an amino acid sequence selected from the group consisting of AYPAAPAPAYPAAPAPYAPA (SEQ ID NO: 1), AQPAAPAPAQPAAPAPQAPA (SEQ ID NO: 2) and ATPAAPAPATPAAPAPTAPA (SEQ ID NO: 3). The amino acid repeats according to the invention do not include any serine residues.

In the context of the present invention, the term “clostridial neurotoxin” refers to a natural neurotoxin obtainable from bacteria of the class Clostridia, including Clostridium tetani and Clostridium botulinum, or to a neurotoxin obtainable from alternative sources, including from recombinant technologies or from genetic or chemical modification. Particularly, the clostridial neurotoxins have endopeptidase activity.

Clostridial neurotoxins are produced as single-chain precursors that are proteolytically cleaved by an unknown clostridial endoprotease within the loop region to obtain the biologically active disulfide-linked di-chain form of the neurotoxin, which comprises two chain elements, a functionally active light chain and a functionally active heavy chain, where one end of the light chain is linked to one end of the heavy chain not via a peptide bond, but via a disulfide bond.

In the context of the present invention, the term “clostridial neurotoxin light chain” refers to that part of a clostridial neurotoxin that comprises an endopeptidase activity responsible for cleaving one or more proteins that is/are part of the so-called SNARE-complex involved in the process resulting in the release of neurotransmitter into the synaptic cleft: In naturally occurring clostridial neurotoxins, the light chain has a molecular weight of approx. 50 kDa.

In the context of the present invention, the term “clostridial neurotoxin heavy chain” refers to that part of a clostridial neurotoxin that is responsible for targeting the cell and entry of the neurotoxin into the neuronal cell: In naturally occurring clostridial neurotoxins, the heavy chain has a molecular weight of approx. 100 kDa.

In the context of the present invention, the term “functionally active clostridial neurotoxin chain” refers to a recombinant clostridial neurotoxin chain able to perform the biological functions of a naturally occurring Clostridium botulinum neurotoxin chain to at least about 25%, particularly to at least about 50%, particularly to at least about 60%, to at least about 70%, to at least about 80%, and most particularly to at least about 90%, where the biological functions of clostridial neurotoxin chains include, but are not limited to, binding of the heavy chain to the neuronal cell, entry of the neurotoxin into a neuronal cell, release of the light chain from the di-chain neurotoxin, and endopeptidase activity of the light chain. Methods for determining a neurotoxic activity can be found, for example, in WO 95/32738, which describes the reconstitution of separately obtained light and heavy chains of tetanus toxin and botulinum toxin. Also cell-based assay methods as described for example in WO2009/114748, WO 2013/049508 and WO2014/207109.

In the context of the present invention, the term “about” or “approximately” means within 20%, alternatively within 10%, including within 5% of a given value or range. Alternatively, especially in biological systems, the term “about” means within about a log (i.e. an order of magnitude), including within a factor of two of a given value.

In the context of the present invention, the term “recombinant clostridial neurotoxin” refers to a composition comprising a clostridial neurotoxin that is obtained by expression of the neurotoxin in a heterologous cell such as E. coli, and including, but not limited to, the raw material obtained from a fermentation process (supernatant, composition after cell lysis), a fraction comprising a clostridial neurotoxin obtained from separating the ingredients of such a raw material in a purification process, an isolated and essentially pure protein, and a formulation for pharmaceutical and/or aesthetic use comprising a clostridial neurotoxin and additionally pharmaceutically acceptable solvents and/or excipients.

In the context of the present invention, the term “recombinant clostridial neurotoxin” further refers to a clostridial neurotoxin based on a parental clostridial neurotoxin additionally comprising a heterologous domain wherein this domain consists of proline and alanine; or proline, alanine and tyrosine; or proline, alanine and glutamine; or proline, alanine and threonine residues; i.e. a domain that is not naturally occurring in said parental clostridial neurotoxin, in particular a synthetic domain, or a domain from a species other than Clostridium botulinum, in particular a domain from a human protein.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components, or groups thereof. The term “comprising” thus includes the more restrictive terms “consisting of” and “consisting essentially of”.

In particular embodiments, said recombinant clostridial neurotoxin exhibits at least one domain comprising an amino acid sequence consisting of between 50 and 500 amino acid residues, more particularly between 70 and 300 amino acid residues, or between 80 and 120 amino acid residues, or between 180 and 220 amino acid residues, particularly 100 amino acid residues, 150 amino acid residues, or 200 amino acid residues.

Surprisingly, it has been found that the attachment of a domain consisting of proline and alanine; or a domain consisting of a plurality of specific amino acid repeats consisting of proline, alanine and tyrosine; or proline, alanine and glutamine; or proline, alanine and threonine residues is able to increase the duration of effect relative to an identical clostridial neurotoxin without said domain. A person skilled in the art would not expect such an effect. It has been shown in WO2015/132004 that the attachment of a neurotoxin with proline, alanine and serine residues could increase the duration of effect, however it was not known that this effect could also be achieved without the amino acid serine. In the context of the present invention the attachment of a domain according to the invention without the amino acid serine surprisingly leads to an increased duration of effect relative to an identical clostridial neurotoxin without said domain.

In particular embodiments, the sequence of said clostridial neurotoxin is selected from the sequence of (i) a Clostridium botulinum neurotoxin serotype A, B, C, D, E, F, and G, or (ii) from the sequence of a functional variant of a Clostridium botulinum neurotoxin of (i).

In the context of the present invention, the term “Clostridium botulinum neurotoxin serotype A, B, C, D, E, F, and G” refers to neurotoxins found in and obtainable from Clostridium botulinum. Currently, seven serologically distinct types, designated serotypes A, B, C, D, E, F, and G are known, including certain subtypes (e.g. A1, A2, A3, A4 and A5).

In particular embodiments, said recombinant clostridial neurotoxin exhibits at least one domain which is inserted at a position selected from (i) the N-terminus of the light chain of said recombinant clostridial neurotoxin; (ii) the C-terminus of the light chain of said recombinant clostridial neurotoxin; (iii) the N-terminus of the heavy chain of said recombinant clostridial neurotoxin; or (iv) the C-terminus of the heavy chain of said recombinant clostridial neurotoxin.

In particular embodiments, said recombinant clostridial neurotoxin comprises two of said domains, wherein both domains comprise an amino acid sequence consisting of at least one proline and at least one alanine residue.

In particular embodiments, said recombinant clostridial neurotoxin comprises two of said domains, wherein both domains comprise an amino acid sequence consisting of a plurality of amino acid repeats, wherein said repeats are selected from the group consisting of AYPAAPAPAYPAAPAPYAPA (SEQ ID NO: 1), AQPAAPAPAQPAAPAPQAPA (SEQ ID NO: 2) and ATPAAPAPATPAAPAPTAPA (SEQ ID NO: 3).

In particular embodiments, said recombinant clostridial neurotoxin comprises two of said domains, wherein one domain is inserted at a position of the N-terminus of the light chain of said recombinant clostridial neurotoxin and the second domain is inserted at a position of the C-terminus of the heavy chain of said recombinant clostridial neurotoxin.

In the context of the present invention, the term “functional variant of a clostridial neurotoxin” refers to a neurotoxin that differs in the amino acid sequence and/or the nucleic acid sequence encoding the amino acid sequence from a clostridial neurotoxin, but is still functionally active. In the context of the present invention, the term “functionally active” refers to the property of a recombinant clostridial neurotoxin to exhibit a biological activity of at least about 20%, particularly to at least about 50%, at least about 70%, at least about 80%, and most particularly at least about 90% of the biological activity of a naturally occurring parental clostridial neurotoxin, i.e. a parental clostridial neurotoxin without said domain, where the biological functions include, but are not limited to, binding to the neurotoxin receptor, entry of the neurotoxin into a neuronal cell, release of the light chain from the di-chain neurotoxin, and endopeptidase activity of the light chain, and thus inhibition of neurotransmitter release from the affected nerve cell. In vivo assays for assessing biological activity include the mouse LD50 assay and the ex vivo mouse hemidiaphragm assay as described by Pearce et al. (Pearce 1994, Toxicol. Appl. Pharmacol. 128: 69-77) and Dressler et al. (Dressler 2005, Mov. Disord. 20:1617-1619, Keller 2006, Neuroscience 139: 629-637) or a cell-based assay as described in WO2009/114748, WO2014/207109 or WO 2013/049508. The biological activity is commonly expressed in Mouse Units (MU). As used herein, 1 MU is the amount of neurotoxic component, which kills 50% of a specified mouse population after intraperitoneal injection, i.e. the mouse i.p. LD50.

On the protein level, a functional variant will maintain key features of the corresponding clostridial neurotoxin, such as key residues for the endopeptidase activity in the light chain, or key residues for the attachment to the neurotoxin receptors or for translocation through the endosomal membrane in the heavy chain, but may contain one or more mutations comprising a deletion of one or more amino acids of the corresponding clostridial neurotoxin, an addition of one or more amino acids of the corresponding clostridial neurotoxin, and/or a substitution of one or more amino acids of the corresponding clostridial neurotoxin. Particularly, said deleted, added and/or substituted amino acids are consecutive amino acids. According to the teaching of the present invention, any number of amino acids may be added, deleted, and/or substituted, as long as the functional variant remains biologically active. For example, 1, 2, 3, 4, 5, up to 10, up to 15, up to 25, up to 50, up to 100, up to 200, up to 400, up to 500 amino acids or even more amino acids may be added, deleted, and/or substituted. Accordingly, a functional variant of the neurotoxin may be a biologically active fragment of a naturally occurring neurotoxin. This neurotoxin fragment may contain an N-terminal, C-terminal, and/or one or more internal deletion(s).

In another embodiment, the functional variant of a clostridial neurotoxin additionally comprises a signal peptide. Usually, said signal peptide will be located at the N-terminus of the neurotoxin. Many such signal peptides are known in the art and are comprised by the present invention. In particular, the signal peptide results in transport of the neurotoxin across a biological membrane, such as the membrane of the endoplasmic reticulum, the Golgi membrane or the plasma membrane of a eukaryotic or prokaryotic cell. It has been found that signal peptides, when attached to the neurotoxin, will mediate secretion of the neurotoxin into the supernatant of the cells. In certain embodiments, the signal peptide will be cleaved off in the course of, or subsequent to, secretion, so that the secreted protein lacks the N-terminal signal peptide, is composed of separate light and heavy chains, which are covalently linked by disulfide bridges, and is proteolytically active.

In particular embodiments, the functional variant has in its clostridium neurotoxin part a sequence identity of at least about 40%, at least about 50%, at least about 60%, at least about 70% or most particularly at least about 80%, and a sequence homology of at least about 60%, at least about 70%, at least about 80%, at least about 90%, or most particularly at least about 95% to the corresponding part in the parental clostridial neurotoxin. Methods and algorithms for determining sequence identity and/or homology, including the comparison of variants having deletions, additions, and/or substitutions relative to a parental sequence, are well known to the practitioner of ordinary skill in the art. The term “identity” as used herein refers to sequence identity characterized by determining the number of identical amino acids between two nucleic acid sequences or two amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wis., USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. On the DNA level, the nucleic acid sequences encoding the functional homologue and the parental clostridial neurotoxin may differ to a larger extent due to the degeneracy of the genetic code. It is known that the usage of codons is different between prokaryotic and eukaryotic organisms. Thus, when expressing a prokaryotic protein such as a clostridial neurotoxin, in a eukaryotic expression system, it may be necessary, or at least helpful, to adapt the nucleic acid sequence to the codon usage of the expression host cell, meaning that sequence identity or homology may be rather low on the nucleic acid level.

In the context of the present invention, the term “variant” refers to a neurotoxin that is a chemically, enzymatically, or genetically modified derivative of a corresponding clostridial neurotoxin, including chemically or genetically modified neurotoxin from C. botulinum, particularly of C. botulinum neurotoxin serotype A, C or E. A chemically modified derivative may be one that is modified by pyruvation, phosphorylation, sulfatation, lipidation, pegylation, glycosylation and/or the chemical addition of an amino acid or a polypeptide comprising between 2 and about 100 amino acids, including modification occurring in the eukaryotic host cell used for expressing the derivative. An enzymatically modified derivative is one that is modified by the activity of enzymes, such as endo- or exoproteolytic enzymes, including modification by enzymes of the eukaryotic host cell used for expressing the derivative. As pointed out above, a genetically modified derivative is one that has been modified by deletion or substitution of one or more amino acids contained in, or by addition of one or more amino acids (including polypeptides comprising between 2 and about 100 amino acids) to, the amino acid sequence of said clostridial neurotoxin. Methods for designing and constructing such chemically or genetically modified derivatives and for testing of such variants for functionality are well known to anyone of ordinary skill in the art.

In particular embodiments, said recombinant clostridial neurotoxin shows an increased duration of effect relative to an identical clostridial neurotoxin without said domain(s).

In the context of the present invention, the term “increased duration of effect” or “increased duration of action” refers to a longer lasting denervation mediated by a clostridial neurotoxin of the present invention. For example, as disclosed herein, administration of a disulfide-linked di-chain clostridial neurotoxin comprising a domain according to the invention results in localized paralysis for a longer period of time relative to administration of an identical disulfide-linked di-chain clostridial neurotoxin without the domain according to the present invention.

In the context of the present invention, the term “increased duration of effect/action” is defined as a more than about 20%, particularly more than about 50%, more particularly more than about 90% increased duration of effect of the recombinant neurotoxin of the present invention relative to the identical neurotoxin without the domain according to the invention.

In the context of the present invention the term “maximum paralytic effect” refers to a value of 80-90% reduction of the initial running distance.

For example, an “increased duration of effect/action” can be determined using the “Mouse Running Assay”. The “Mouse Running Assay” is well-known to the person skilled in the art and measures the daily running distance of a mouse in a treadmill after a botulinum neurotoxin was injected into the M. gastrocnemius (see Keller J E. Recovery from botulinum neurotoxin poisoning in vivo. Neuroscience. 2006 May 12; 139(2):629-37). The distance which a mouse is able to run in the treadmill the day before the botulinum neurotoxin is injected is used as comparison and is set as 100%. A daily running distance of no more than 80% of the initial running distance is regarded as paralysis of the muscle. The duration of effect is determined by the time period between the time point attaining a half-maximal paralysis, i.e. about 40% of the initial running distance, and the time point when paralysis reaches recovery, i.e. about 40% of the initial running distance. If this time period is 2 days longer compared with the standard (wildtype BoNT) provided that the mutated BoNT exhibits a similar potency i.e shows a similar maximal paralysis (reduction of the running distance) of about 80-90%, the botulinum neurotoxin is considered to exhibit an “increased duration of effect/action”.

In the context of the present invention the term “denervation” refers to denervation resulting from administration of a chemodenervating agent, for example a neurotoxin.

In the context of the present invention, the term “localized denervation” or “localized paralysis” refers to denervation of a particular anatomical region, usually a muscle or a group of anatomically and/or physiologically related muscles, which results from administration of a chemodenervating agent, for example a neurotoxin, to the particular anatomical region.

Without wishing to be bound by theory, the recombinant clostridial neurotoxins of the present invention might show increased biological half-life, reduced degradation rates, decreased diffusion rates, increased uptake by neuronal cells, and/or modified intracellular translocation rates, in each case relative to an identical parental clostridial neurotoxin without the domain according to the invention.

In another aspect, the present invention relates to a pharmaceutical or cosmetic composition comprising the recombinant clostridial neurotoxin of the present invention. For preparing a pharmaceutical preparation comprising a clostridial neurotoxin the toxin can be formulated by various techniques dependent on the desired application purposes which are known in the art. For example, the (biologically active) botulinum neurotoxin polypeptide can be used in combination with one or more pharmaceutically acceptable carriers as a pharmaceutical composition. The pharmaceutically acceptable carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatine, agar, pectin, acacia, magnesium stearate, stearic acid and the like. Exemplary of liquid carriers are glycerol, phosphate buffered saline solution, water, emulsions, various types of wetting agents, and the like. Suitable carriers comprise those mentioned above and others well known in the art, see, e.g., Remington's Pharmaceutical Sciences, Mack Publishing Company, Easton, Pa. In an aspect, the pharmaceutical composition can be dissolved in a diluent, prior to administration. The diluent is also selected so as not to affect the biological activity of the Neurotoxin product. Examples of such diluents are distilled water or physiological saline. In addition, the pharmaceutical composition or formulation may also include other carriers or non-toxic, non-therapeutic, non-immunogenic stabilizers and the like. Thus, the formulated Neurotoxin product can be present, in an aspect, in liquid or lyophilized form. In an aspect, it can be present together with glycerol, protein stabilizers (HSA) or non-protein stabilizers such as polyvinyl pyrrolidone (PVP), hyaluronic acid, polysorbate or free amino acids. In an aspect, suitable non-proteinaceous stabilizers are disclosed in WO 2005/007185 or WO 2006/020208. The formulated Neurotoxin product may be used for human or animal therapy of various diseases or disorders in a therapeutically effective dose or for cosmetic purposes.

In particular embodiments, the recombinant clostridial neurotoxin of the present invention or the pharmaceutical composition of the present invention is for use in the treatment of a disease or condition taken from the list of: cervical dystonia (spasmodic torticollis), blepharospasm, severe primary axillary hyperhidrosis, achalasia, lower back pain, benign prostate hypertrophy, chronic focal painful neuropathies, migraine and other headache disorders.

Additional indications where treatment with botulinum neurotoxins is currently under investigation and where the pharmaceutical composition of the present invention may be used, include pediatric incontinence, incontinence due to overactive bladder, and incontinence due to neurogenic bladder, anal fissure, spastic disorders associated with injury or disease of the central nervous system including trauma, stroke, multiple sclerosis, Parkinson's disease, or cerebral palsy, focal dystonias affecting the limbs, face, jaw or vocal cords, temporomandibular joint (TMJ) pain disorders, diabetic neuropathy, wound healing, excessive salivation, vocal cord dysfunction, reduction of the Masseter muscle for decreasing the size of the lower jaw, treatment and prevention of chronic headache and chronic musculoskeletal pain, treatment of snoring noise, assistance in weight loss by increasing the gastric emptying time.

Most recently, clostridial neurotoxins have been evaluated for the treatment of other new indications, for example painful keloid, diabetic neuropathic pain, refractory knee pain, trigeminal neuralgia trigger-zone application to control pain, scarring after cleft-lip surgery, cancer and depression.

In yet another aspect, the present invention relates to the use of the composition of the present invention for cosmetic treatment.

Thus, in another aspect, the present invention relates to a method of cosmetically treating a patient, comprising the step of administering a composition comprising a recombinant clostridial neurotoxin according to the present invention to a patient desiring such cosmetic treatment.

In the context of the present invention, the term “cosmetic treatment” relates to uses in cosmetic or aesthetic applications, such as the treatment of wrinkles, crow's feet, glabella frown lines, reduction of the masseter muscle, reduction of the calves, removing of facial asymmetries etc.

In another aspect, the present invention relates to a method for the generation of the recombinant clostridial neurotoxin of the present invention, comprising the step of obtaining a recombinant nucleic acid sequence encoding a recombinant single-chain precursor clostridial neurotoxin by the insertion of a nucleic acid sequence encoding said domain according to the invention into a nucleic acid sequence encoding a parental clostridial neurotoxin.

In the context of the present invention, the term “recombinant nucleic acid sequence” refers to a nucleic acid, which has been generated by joining genetic material from two different sources.

In the context of the present invention, the term “single-chain precursor clostridial neurotoxin” refers to a single-chain precursor for a disulfide-linked di-chain clostridial neurotoxin, comprising a functionally active clostridial neurotoxin light chain, a functionally active neurotoxin heavy chain, and a loop region linking the C-terminus of the light chain with the N-terminus of the heavy chain.

In the context of the present invention, the term “recombinant single-chain precursor clostridial neurotoxin” refers to a single-chain precursor clostridial neurotoxin comprising a heterologous domain, i.e. a domain from a species other than Clostridium botulinum.

In particular embodiments, the recombinant single-chain precursor clostridial neurotoxin comprises a protease cleavage site in said loop region.

Single-chain precursor clostridial neurotoxins have to be proteolytically cleaved to obtain the final biologically active clostridial neurotoxins. Proteolytic cleavage may either occur during heterologous expression by host cell enzymes, or by adding proteolytic enzymes to the raw protein material isolated after heterologous expression. Naturally occurring clostridial neurotoxins usually contain one or more cleavage signals for proteases which post-translationally cleave the single-chain precursor molecule, so that the final di- or multimeric complex can form. At present, clostridial neurotoxins are still predominantly produced by fermentation processes using appropriate Clostridium strains. During the fermentation process, the single-chain precursors are proteolytically cleaved by an unknown clostridial protease to obtain the biologically active di-chain clostridial neurotoxin. In cases, where the single-chain precursor molecule is the precursor of a protease, autocatalytic cleavage may occur. Alternatively, the protease can be a separate non-clostridial enzyme expressed in the same cell. WO 2006/076902 describes the proteolytic cleavage of a recombinant clostridial neurotoxin single-chain precursor at a heterologous recognition and cleavage site by incubation of the E. coli host cell lysate. The proteolytic cleavage is carried out by an unknown E. coli protease. In certain applications of recombinant expression, modified protease cleavage sites have been introduced recombinantly into the interchain region between the light and heavy chain of clostridial toxins, e.g. protease cleavage sites for human thrombin or non-human proteases (see WO 01/14570).

In particular embodiments, the protease cleavage site is a site that is cleaved by a protease selected from the list of: thrombin, trypsin, enterokinase, factor Xa, plant papain, insect papain, crustacean papain, enterokinase, human rhinovirus 3C protease, human enterovirus 3C protease, tobacco etch virus protease, Tobacco Vein Mottling Virus, subtilisin and caspase 3.

In a particular embodiment, the recombinant single-chain precursor clostridial neurotoxin further comprises a binding tag, particularly selected from the group comprising: glutathione-S-transferase (GST), maltose binding protein (MBP), a His-tag, a Strep-tag, or a FLAG-tag.

In the context of the present invention, the term “parental clostridial neurotoxin” refers to an initial clostridial neurotoxin without a heterologous domain according to the invention, selected from a natural clostridial neurotoxin, a functional variant of a natural clostridial neurotoxin or a chimeric clostridial neurotoxin, wherein the clostridial neurotoxin light chain and heavy chain are from different clostridial neurotoxin serotypes.

In particular embodiments, the method for the generation of the recombinant clostridial neurotoxin of the present invention further comprises the step of heterologously expressing said recombinant nucleic acid sequence in a host cell, particularly in a bacterial host cell, more particularly in an E. coli host cell.

In certain embodiments, the E. coli cells are selected from E. coli XL1-Blue, Nova Blue, TOP10, XL10-Gold, BL21, and K12.

In particular embodiments, the method for the generation of the recombinant clostridial neurotoxin of the present invention additionally comprises at least one of the steps of (i) generating a disulfide-linked di-chain recombinant clostridial neurotoxin comprising said domain according to the invention by causing or allowing contacting of said recombinant single-chain precursor clostridial neurotoxin with an endoprotease and (ii) purification of said recombinant single-chain precursor clostridial neurotoxin or said disulfide-linked di-chain recombinant clostridial neurotoxin by chromatography.

In particular embodiments, the recombinant single-chain precursor clostridial neurotoxin, or the recombinant disulfide-linked di-chain clostridial neurotoxin, is purified after expression, or in the case of the recombinant disulfide-linked di-chain clostridial neurotoxin, after the cleavage reaction. In particular such embodiments, the protein is purified by chromatography, particularly by immunoaffinity chromatography, or by chromatography on an ion exchange matrix, a hydrophobic interaction matrix, or a multimodal chromatography matrix, particularly a strong ion exchange matrix, more particularly a strong cation exchange matrix.

In the context of the present invention, the term “causing . . . contacting of said recombinant single-chain precursor clostridial neurotoxin . . . with an endoprotease” refers to an active and/or direct step of bringing said neurotoxin and said endoprotease in contact, whereas the term “allowing contacting of a recombinant single-chain precursor clostridial neurotoxin . . . with an endoprotease” refers to an indirect step of establishing conditions in such a way that said neurotoxin and said endoprotease are getting in contact to each other.

In the context of the present invention, the term “endoprotease” refers to a protease that breaks peptide bonds of non-terminal amino acids (i.e. within the polypeptide chain). As they do not attack terminal amino acids, endoproteases cannot break down peptides into monomers.

In particular embodiments, cleavage of the recombinant single-chain precursor clostridial neurotoxin is near-complete.

In the context of the present invention, the term “near-complete” is defined as more than about 95% cleavage, particularly more than about 97.5%, more particularly more than about 99% as determined by SDS-PAGE and subsequent Western Blot or reversed phase chromatography.

In particular embodiments, cleavage of the recombinant single-chain precursor clostridial neurotoxin occurs at a heterologous cleavage signal located in the loop region of the recombinant precursor clostridial neurotoxin.

In particular embodiments, the cleavage reaction is performed with crude host cell lysates containing said single-chain precursor protein.

In other particular embodiments, the single-chain precursor protein is purified or partially purified, particularly by a first chromatographic enrichment step, prior to the cleavage reaction.

In the context of the present invention, the term “purified” relates to more than about 90% purity. In the context of the present invention, the term “partially purified” relates to purity of less than about 90% and an enrichment of more than about two fold.

In another aspect, the present invention relates to a recombinant single-chain clostridial neurotoxin, which is a precursor for the recombinant clostridial neurotoxin of the present invention Thus, in such aspect, the present invention relates to a recombinant single-chain precursor clostridial neurotoxin comprising at least one domain according to the invention.

In another aspect, the present invention relates to a nucleic acid sequence encoding the recombinant single-chain clostridial neurotoxin of the present invention.

In another aspect, the present invention relates to a method for obtaining the nucleic acid sequence of the present invention, comprising the step of inserting a nucleic acid sequence encoding said domain into a nucleic acid sequence encoding a parental clostridial neurotoxin.

In another aspect, the present invention relates to a vector comprising the nucleic acid sequence of the present invention, or the nucleic acid sequence obtainable by the method of the present invention.

In another aspect, the present invention relates to a recombinant host cell comprising the nucleic acid sequence of the present invention, the nucleic acid sequence obtainable by the method of the present invention, or the vector of the present invention.

In another aspect, the present invention relates to a method for producing the recombinant single-chain precursor clostridial neurotoxin of the present invention, comprising the step of expressing the nucleic acid sequence of the present invention, or the nucleic acid sequence obtainable by the method of the present invention, or the vector of the present invention in a recombinant host cell, or cultivating the recombinant host cell of the present invention under conditions that result in the expression of said nucleic acid sequence.

### EXAMPLES

### Example 1: Generation and Purification of a PA100-rBoNT/A-PA100

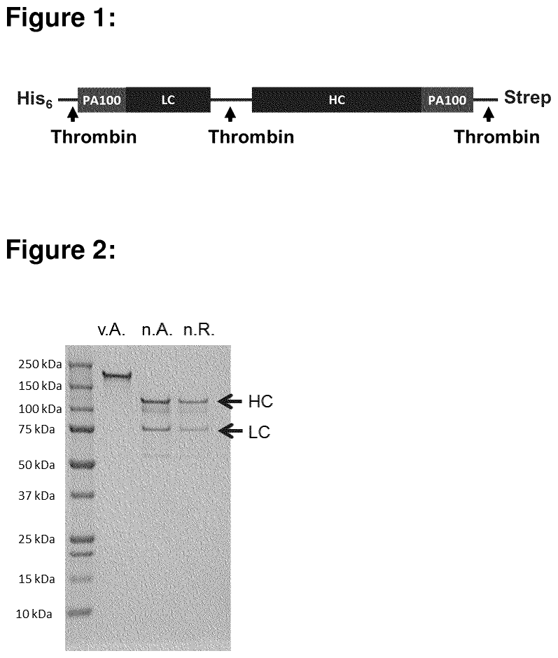

The nucleic acid construct encoding two “PA” modules comprising each additional 100 amino acid residues respectively ((AAPAA PAPAA PAAPA PAAPA)₅) built from the amino acids proline and alanine was synthetically produced. By using restriction enzymes NdeI and SwaI as well as BglII and AatII the corresponding gene module PA100 was inserted at the N-terminus and C-terminus of recombinant BoNT/A (PA100-rBoNT/A-PA100), wherein the linker exhibited a thrombin cleavage site sequence (FIG. 1). The correct cloning was verified by sequencing.

Expression was performed in expression strain E. coli Bl21. Purification was done using a combination of his affinity, ion exchange and size exclusion chromatography, followed by activation using thrombin. FIG. 2 summarizes the results of purification and activation.

### Example 2: Duration of Effect of PA100-rBoNT/A-PA100 in a “Mouse Running Assay”

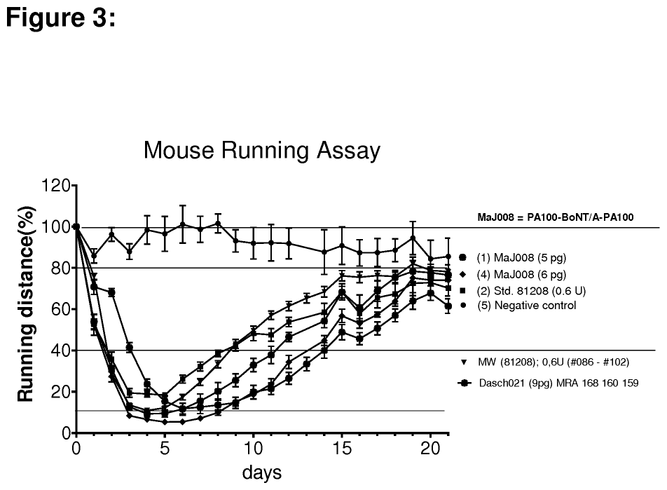

Two different dosages of PA100-rBoNT/A-PA100 (5, 6 pg) were injected into the M. gastrocnemius of eight mice in comparison to standard Xeomin® (Std. 81208; 0.6 U), a mean of standard (17 assays) of Xeomin® 81208 (0.6 U) and a dosage of a different modified BoNT PAS100-rBoNT/A-PAS100 (9 pg) having two “PAS” modules each comprising 100 amino acid residues built from the amino acids proline, alanine and serine. 5 pg of PA100-rBoNT/A-PA100 eliciting a similar maximal reduction in the running distance was equipotent to Xeomin®. The mice had been trained in a treadmill. The daily running distance in the treadmill was measured over 21 days. The paralysis caused by the toxins was plotted as percentage of the running distance on the day before the injection, which was set as 100%, against the time (see FIG. 3).

During the recovery phase the running distance of the control group (mean of standard (17 assays) of Xeomin®) reached a value of 40% of the starting value 7 days after half-maximum paralysis was observed (day 9), whereas the group treated with 5 pg PA100-rBoNT/A-PA100 reached that value 9 days after half-maximum paralysis (day 11). Thus, the duration of effective paralysis was significantly extended.

### Example 3: Generation and Purification of a PAY100-rBoNT/A-PAY100

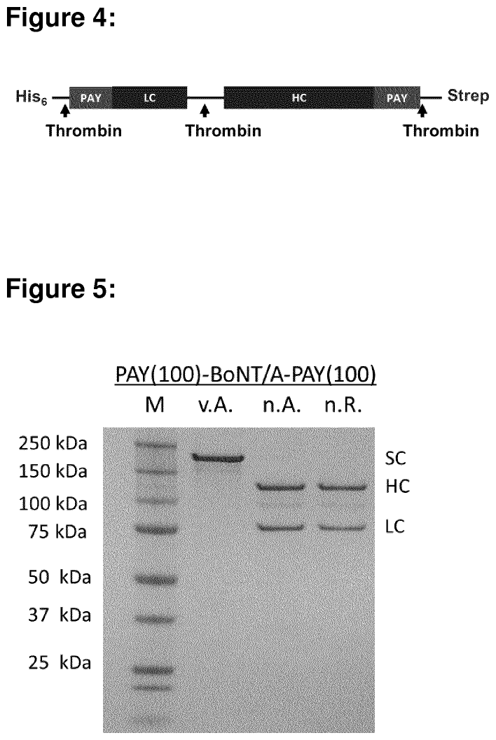

The nucleic acid construct encoding two “PAY” modules comprising each additional 100 amino acid residues respectively built from the amino acids repeats consisting of AYPAAPAPAYPAAPAPYAPA (SEQ ID NO: 1) was synthetically produced. By using suitable restriction enzymes the corresponding gene module PAY100 was inserted at the N-terminus and C-terminus of recombinant BoNT/A (PAY100-rBoNT/A-PAY100), wherein the linker exhibited a thrombin cleavage site sequence (FIG. 4). The correct cloning was verified by sequencing.

Expression was performed in expression strain E. coli Bl21. Purification was done using a combination of his affinity, ion exchange and size exclusion chromatography, followed by activation using thrombin. FIG. 5 summarizes the results of purification and activation.

### Example 4: Duration of Effect of PAY100-rBoNT/A-PAY100 in a “Mouse Running Assay”

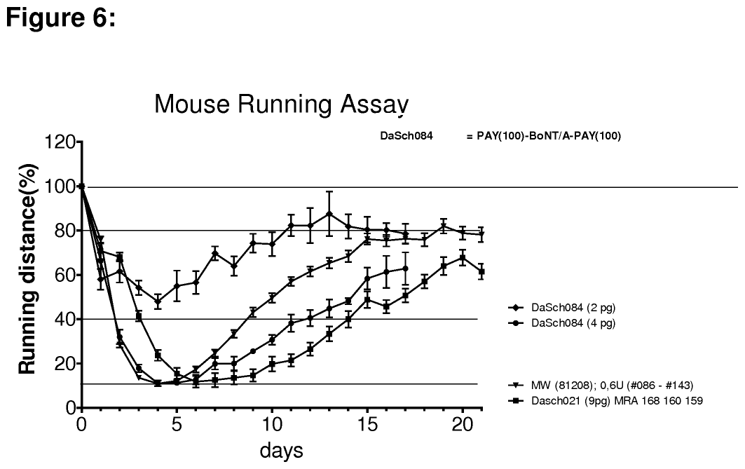

Two dosages of PAY100-rBoNT/A-PAY100 (2, 4 pg) were injected into the M. gastrocnemius of eight mice in comparison to a mean of standard (17 assays) of Xeomin® 81208 (0.6 U) and to a dosage of a different modified BoNT PAS100-rBoNT/A-PAS100 (9 pg) having two “PAS” modules each comprising 100 amino acid residues built from the amino acids proline, alanine and serine. 4 pg of PAY100-rBoNT/A-PAY100 eliciting a similar maximal reduction in the running distance was equipotent to Xeomin®. The mice had been trained in a treadmill. The daily running distance in the treadmill was measured over 21 days. The paralysis caused by the toxins was plotted as percentage of the running distance on the day before the injection, which was set as 100%, against the time (see FIG. 6).

During the recovery phase the running distance of the control group (mean of standard (17 assays) of Xeomin®) reached a value of 40% of the starting value 7 days after half-maximum paralysis was observed (day 9), whereas the group treated with 4 pg PAY100-rBoNT/A-PAY100 reached that value 10 days after half-maximum paralysis (day 12). Thus, the duration of effective paralysis was significantly extended.

### Example 5: Generation and Purification of a PAQ100-rBoNT/A-PAQ100

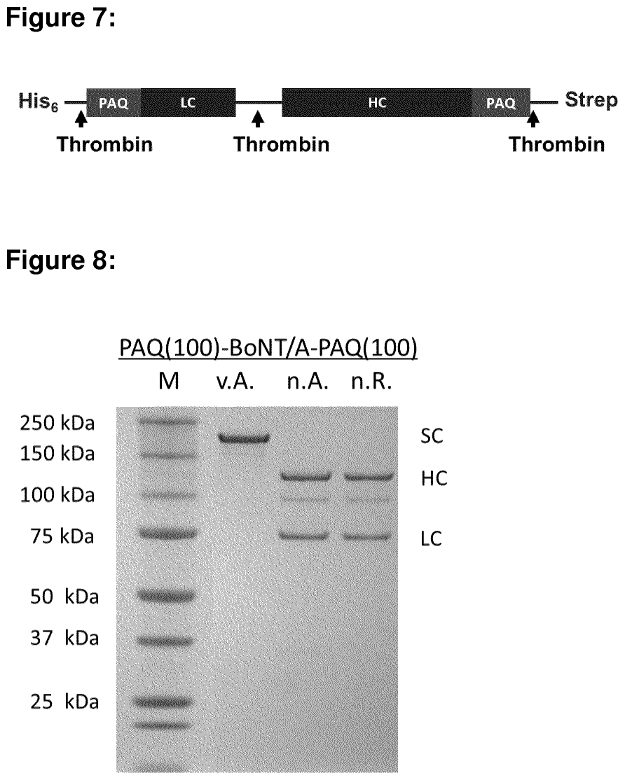

The nucleic acid construct encoding two “FAQ” modules comprising each additional 100 amino acid residues respectively built from the amino acids repeats consisting of AQPAAPAPAQPAAPAPQAPA (SEQ ID NO: 2) was synthetically produced. By using suitable restriction enzymes the corresponding gene module PAQ100 was inserted at the N-terminus and C-terminus of recombinant BoNT/A (PAQ100-rBoNT/A-PAQ100), wherein the linker exhibited a thrombin cleavage site sequence (FIG. 7). The correct cloning was verified by sequencing.

Expression was performed in expression strain E. coli Bl21. Purification was done using a combination of his affinity, ion exchange and size exclusion chromatography, followed by activation using thrombin. FIG. 8 summarizes the results of purification and activation.

### Example 6: Duration of Effect of PAQ100-rBoNT/A-PAQ100 in a “Mouse Running Assay”

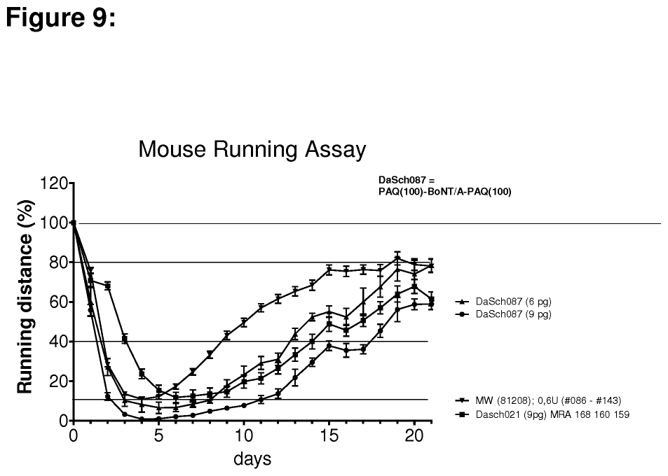

Two dosages of PAQ100-rBoNT/A-PAQ100 (6, 9 pg) were injected into the M. gastrocnemius of eight mice in comparison to a mean of standard (17 assays) of Xeomin® 81208 (0.6 U) and to a dosage of a different modified BoNT PAS100-rBoNT/A-PAS100 (9 pg) having two “PAS” modules each comprising 100 amino acid residues built from the amino acids proline, alanine and serine. 6 pg of PAQ100-rBoNT/A-PAQ100 eliciting a similar maximal reduction in the running distance was equipotent to Xeomin®. The mice had been trained in a treadmill. The daily running distance in the treadmill was measured over 21 days. The paralysis caused by the toxins was plotted as percentage of the running distance on the day before the injection, which was set as 100%, against the time (see FIG. 9).

During the recovery phase the running distance of the control group (mean of standard (17 assays) of Xeomin®) reached a value of 40% of the starting value 7 days after half-maximum paralysis was observed (day 9), whereas the group treated with 6 pg PAQ100-rBoNT/A-PAQ100 reached that value 10 days after half-maximum paralysis (day 12). Thus, the duration of effective paralysis was significantly extended.

### Example 7: Generation and Purification of a PAT100-rBoNT/A-PAT100

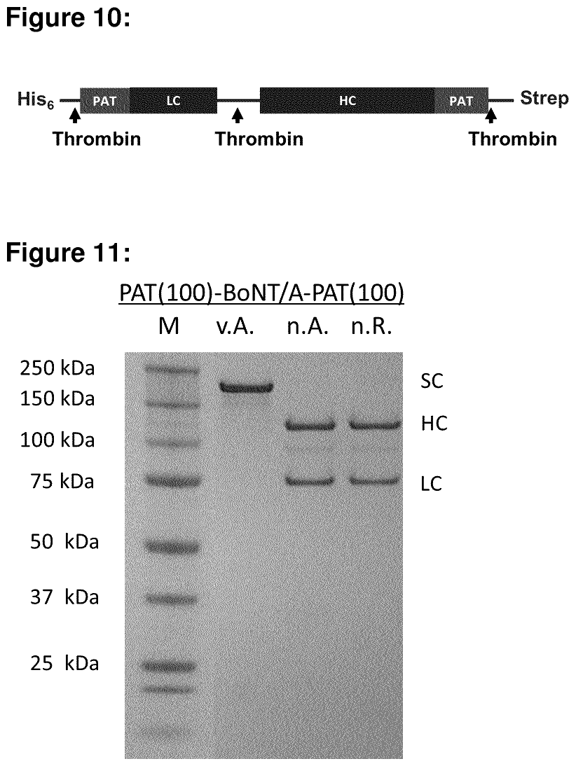

The nucleic acid construct encoding two “PAT” modules comprising each additional 100 amino acid residues respectively built from the amino acids repeats consisting of ATPAAPAPATPAAPAPTAPA (SEQ ID NO: 3) was synthetically produced. By using suitable restriction enzymes the corresponding gene module PAT100 was inserted at the N-terminus and C-terminus of recombinant BoNT/A (PAT100-rBoNT/A-PAT100), wherein the linker exhibited a thrombin cleavage site sequence (FIG. 10). The correct cloning was verified by sequencing.

Expression was performed in expression strain E. coli Bl21. Purification was done using a combination of his affinity, ion exchange and size exclusion chromatography, followed by activation using thrombin. FIG. 11 summarizes the results of purification and activation.

### Example 8: Duration of Effect of PAT100-rBoNT/A-PAT100 in a “Mouse Running Assay”

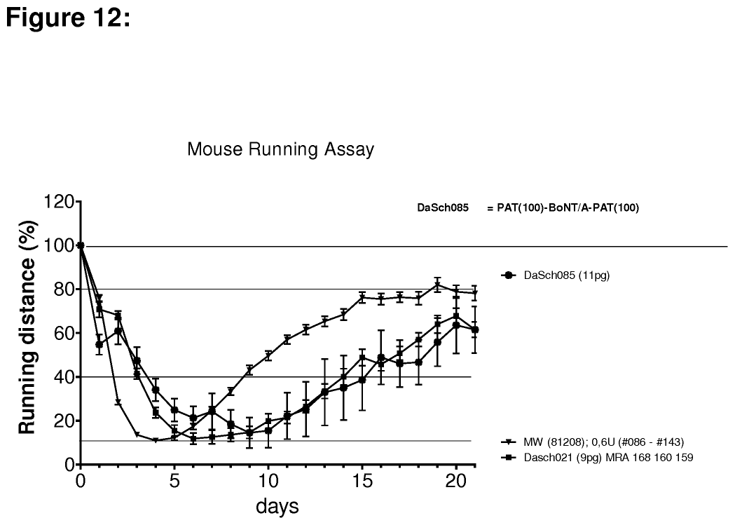

A dosage of PAT100-rBoNT/A-PAT100 (11 pg) was injected into the M. gastrocnemius of eight mice in comparison to a mean of standard (17 assays) of Xeomin® 81208 (0.6 U) and to a dosage of a different modified BoNT PAS100-rBoNT/A-PAS100 (9 pg; Dasch021) having two “PAS” modules each comprising 100 amino acid residues built from the amino acids proline, alanine and serine. 11 pg of PAT100-rBoNT/A-PAT100 eliciting a similar maximal reduction in the running distance was equipotent to Xeomin®. The mice had been trained in a treadmill. The daily running distance in the treadmill was measured over 21 days. The paralysis caused by the toxins was plotted as percentage of the running distance on the day before the injection, which was set as 100%, against the time (see FIG. 12).

During the recovery phase the running distance of the control group (mean of standard (17 assays) of Xeomin®) reached a value of 40% of the starting value 7 days after half-maximum paralysis was observed (day 9), whereas the group treated with 11 pg PAT100-rBoNT/A-PAT100 reached that value about 11 days after half-maximum paralysis (day 15). Thus, the duration of effective paralysis was significantly extended.

TABLE 1
Sequences

SEQ ID NO 1 (PAY100)
[AYPAAPAPAYPAAPAPYAPA]₅
SEQ ID NO 2: (PAQ100)
[AQPAAPAPAQPAAPAPQAPA]₅
SEQ ID NO 3: (PAT100)
[ATPAAPAPATPAAPAPTAPA]₅
SEQ ID NO 4: (PA100-rBoNTA-PA100)
MGSSHHHHHHGSLVPRSSSAAPAAPAPAAPAAPAPAAPAAAPAAPAPAAPAAPAPAAPAA
APAAPAPAAPAAPAPAAPAAAPAAPAPAAPAAPAPAAPAAAPAAPAPAAPAAPAPAAPAP
FVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGST
IDTELKVIDTNCINVIQPDGSYRSEELNLVIIGPSADIIQFECKSFGHEVLNLTRNGYGS
TQYIRFSPDFTFGFEESLEVDTNPLLGAGKFATDPAVTLAHELIHAGHRLYGIAINPNRV
FKVNTNAYYEMSGLEVSFEELRTFGGHDAKFIDSLQENEFRLYYYNKFKDIASTLNKAKS
IVGTTASLQYMKNVFKEKYLLSEDTSGKFSVDKLKFDKLYKMLTEIYTEDNFVKFFKVLN
RKTYLNFDKAVFKINIVPKVNYTIYDGFNLRNTNLAANFNGQNTEINNMNFTKLKNFTGL
FEFYKLLCVRGIITSKAGAGKSLVPRGSAGAGALNDLCIKVNNWDLFFSPSEDNFTNDLN
KGEEITSDTNIEAAEENISLDLIQQYYLTFNFDNEPENISIENLSSDIIGQLELMPNIER
FPNGKKYELDKYTMFHYLRAQEFEHGKSRIALTNSVNEALLNPSRVYTFFSSDYVKKVNK
ATEAAMFLGWVEQLVYDFTDETSEVSTTDKIADITIIIPYIGPALNIGNMLYKDDFVGAL
IFSGAVILLEFIPEIAIPVLGTFALVSYIANKVLTVQTIDNALSKRNEKWDEVYKYIVTN
WLAKVNTQIDLIRKKMKEALENQAEATKAIINYQYNQYTEEEKNNINFNIDDLSSKLNES
INKAMININKFLNQCSVSYLMNSMIPYGVKRLEDFDASLKDALLKYIYDNRGTLIGQVDR
LKDKVNNTLSTDIPFQLSKYVDNQRLLSTFTEYIKNIINTSILNLRYESNHLIDLSRYAS
KINIGSKVNFDPIDKNQIQLFNLESSKIEVILKNAIVYNSMYENFSTSFWIRIPKYFNSI
SLNNEYTIINCMENNSGWKVSLNYGEIIWTLQDTQEIKQRVVFKYSQMINISDYINRWIF
VTITNNRLNNSKIYINGRLIDQKPISNLGNIHASNNIMFKLDGCRDTHRYIWIKYFNLFD
KELNEKEIKDLYDNQSNSGILKDFWGDYLQYDKPYYMLNLYDPNKYVDVNNVGIRGYMYL
KGPRGSVMTTNIYLNSSLYRGTKFIIKKYASGNKDNIVRNNDRVYINVVVKNKEYRLATN
ASQAGVEKILSALEIPDVGNLSQVVVMKSKNDQGITNKCKMNLQDNNGNDIGFIGFHQFN
NIAKLVASNWYNRQIERSSRTLGCSWEFIPVDDGWGERPLAAPAAPAPAAPAAPAPAAPA
AAPAAPAPAAPAAPAPAAPAAAPAAPAPAAPAAPAPAAPAAAPAAPAPAAPAAPAPAAPA
AAPAAPAPAAPAAPAPAAPAGDLVPRGSANSSSVDKLWSHPQFEK
SEQ ID NO 5: (PAY100-rBoNTA-PAY100)
MGSSHHHHHHGSLVPRSSSAYPAAPAPAYPAAPAPYAPAAYPAAPAPAYPAAPAPYAPAA
YPAAPAPAYPAAPAPYAPAAYPAAPAPAYPAAPAPYAPAAYPAAPAPAYPAAPAPYAPAP
FVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGST
IDTELKVIDTNCINVIQPDGSYRSEELNLVIIGPSADIIQFECKSFGHEVLNLTRNGYGS
TQYIRFSPDFTFGFEESLEVDTNPLLGAGKFATDPAVTLAHELIHAGHRLYGIAINPNRV
FKVNTNAYYEMSGLEVSFEELRTFGGHDAKFIDSLQENEFRLYYYNKFKDIASTLNKAKS
IVGTTASLQYMKNVFKEKYLLSEDTSGKFSVDKLKFDKLYKMLTEIYTEDNFVKFFKVLN
RKTYLNFDKAVFKINIVPKVNYTIYDGFNLRNTNLAANFNGQNTEINNMNFTKLKNFTGL
FEFYKLLCVRGIITSKAGAGKSLVPRGSAGAGALNDLCIKVNNWDLFFSPSEDNFTNDLN
KGEEITSDTNIEAAEENISLDLIQQYYLTFNFDNEPENISIENLSSDIIGQLELMPNIER
FPNGKKYELDKYTMFHYLRAQEFEHGKSRIALTNSVNEALLNPSRVYTFFSSDYVKKVNK
ATEAAMFLGWVEQLVYDFTDETSEVSTTDKIADITIIIPYIGPALNIGNMLYKDDFVGAL
IFSGAVILLEFIPEIAIPVLGTFALVSYIANKVLTVQTIDNALSKRNEKWDEVYKYIVTN
WLAKVNTQIDLIRKKMKEALENQAEATKAIINYQYNQYTEEEKNNINFNIDDLSSKLNES
INKAMININKFLNQCSVSYLMNSMIPYGVKRLEDFDASLKDALLKYIYDNRGTLIGQVDR
LKDKVNNTLSTDIPFQLSKYVDNQRLLSTFTEYIKNIINTSILNLRYESNHLIDLSRYAS
KINIGSKVNFDPIDKNQIQLFNLESSKIEVILKNAIVYNSMYENFSTSFWIRIPKYFNSI
SLNNEYTIINCMENNSGWKVSLNYGEIIWTLQDTQEIKQRVVFKYSQMINISDYINRWIF
VTITNNRLNNSKIYINGRLIDQKPISNLGNIHASNNIMFKLDGCRDTHRYIWIKYFNLFD
KELNEKEIKDLYDNQSNSGILKDFWGDYLQYDKPYYMLNLYDPNKYVDVNNVGIRGYMYL
KGPRGSVMTTNIYLNSSLYRGTKFIIKKYASGNKDNIVRNNDRVYINVVVKNKEYRLATN
ASQAGVEKILSALEIPDVGNLSQVVVMKSKNDQGITNKCKMNLQDNNGNDIGFIGFHQFN
NIAKLVASNWYNRQIERSSRTLGCSWEFIPVDDGWGERPLAAYPAAPAPAYPAAPAPYAP
AAYPAAPAPAYPAAPAPYAPAAYPAAPAPAYPAAPAPYAPAAYPAAPAPAYPAAPAPYAP
AAYPAAPAPAYPAAPAPQAPAGDLVPRGSANSSSVDKLWSHPQFEK
SEQ ID NO 6: (PAQ100-rBoNTA-PAQ100)
MGSSHHHHHHGSLVPRSSSAQPAAPAPAQPAAPAPQAPAAQPAAPAPAQPAAPAPQAPAA
QPAAPAPAQPAAPAPQAPAAQPAAPAPAQPAAPAPQAPAAQPAAPAPAQPAAPAPQAPAP
FVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGST
IDTELKVIDTNCINVIQPDGSYRSEELNLVIIGPSADIIQFECKSFGHEVLNLTRNGYGS
TQYIRFSPDFTFGFEESLEVDTNPLLGAGKFATDPAVTLAHELIHAGHRLYGIAINPNRV
FKVNTNAYYEMSGLEVSFEELRTFGGHDAKFIDSLQENEFRLYYYNKFKDIASTLNKAKS
IVGTTASLQYMKNVFKEKYLLSEDTSGKFSVDKLKFDKLYKMLTEIYTEDNFVKFFKVLN
RKTYLNFDKAVFKINIVPKVNYTIYDGFNLRNTNLAANFNGQNTEINNMNFTKLKNFTGL
FEFYKLLCVRGIITSKAGAGKSLVPRGSAGAGALNDLCIKVNNWDLFFSPSEDNFTNDLN
KGEEITSDTNIEAAEENISLDLIQQYYLTFNFDNEPENISIENLSSDIIGQLELMPNIER
FPNGKKYELDKYTMFHYLRAQEFEHGKSRIALTNSVNEALLNPSRVYTFFSSDYVKKVNK
ATEAAMFLGWVEQLVYDFTDETSEVSTTDKIADITIIIPYIGPALNIGNMLYKDDFVGAL
IFSGAVILLEFIPEIAIPVLGTFALVSYIANKVLTVQTIDNALSKRNEKWDEVYKYIVTN
WLAKVNTQIDLIRKKMKEALENQAEATKAIINYQYNQYTEEEKNNINFNIDDLSSKLNES
INKAMININKFLNQCSVSYLMNSMIPYGVKRLEDFDASLKDALLKYIYDNRGTLIGQVDR
LKDKVNNTLSTDIPFQLSKYVDNQRLLSTFTEYIKNIINTSILNLRYESNHLIDLSRYAS
KINIGSKVNFDPIDKNQIQLFNLESSKIEVILKNAIVYNSMYENFSTSFWIRIPKYFNSI
SLNNEYTIINCMENNSGWKVSLNYGEIIWTLQDTQEIKQRVVFKYSQMINISDYINRWIF
VTITNNRLNNSKIYINGRLIDQKPISNLGNIHASNNIMFKLDGCRDTHRYIWIKYFNLFD
KELNEKEIKDLYDNQSNSGILKDFWGDYLQYDKPYYMLNLYDPNKYVDVNNVGIRGYMYL
KGPRGSVMTTNIYLNSSLYRGTKFIIKKYASGNKDNIVRNNDRVYINVVVKNKEYRLATN
ASQAGVEKILSALEIPDVGNLSQVVVMKSKNDQGITNKCKMNLQDNNGNDIGFIGFHQFN
NIAKLVASNWYNRQIERSSRTLGCSWEFIPVDDGWGERPLAAQPAAPAPAQPAAPAPQAP
AAQPAAPAPAQPAAPAPQAPAAQPAAPAPAQPAAPAPQAPAAQPAAPAPAQPAAPAPQAP
AAQPAAPAPAQPAAPAPQAPAGDLVPRGSANSSSVDKLWSHPQFEK
SEQ ID NO 7: (PAT100-rBoNTA-PAT100)
MGSSHHHHHHGSLVPRSSSATPAAPAPATPAAPAPTAPAATPAAPAPATPAAPAPTAPAA
TPAAPAPATPAAPAPTAPAATPAAPAPATPAAPAPTAPAATPAAPAPATPAAPAPTAPAP
FVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGST
IDTELKVIDTNCINVIQPDGSYRSEELNLVIIGPSADIIQFECKSFGHEVLNLTRNGYGS
TQYIRFSPDFTFGFEESLEVDTNPLLGAGKFATDPAVTLAHELIHAGHRLYGIAINPNRV
FKVNTNAYYEMSGLEVSFEELRTFGGHDAKFIDSLQENEFRLYYYNKFKDIASTLNKAKS
IVGTTASLQYMKNVFKEKYLLSEDTSGKFSVDKLKFDKLYKMLTEIYTEDNFVKFFKVLN
RKTYLNFDKAVFKINIVPKVNYTIYDGFNLRNTNLAANFNGQNTEINNMNFTKLKNFTGL
FEFYKLLCVRGIITSKAGAGKSLVPRGSAGAGALNDLCIKVNNWDLFFSPSEDNFTNDLN
KGEEITSDTNIEAAEENISLDLIQQYYLTFNFDNEPENISIENLSSDIIGQLELMPNIER
FPNGKKYELDKYTMFHYLRAQEFEHGKSRIALTNSVNEALLNPSRVYTFFSSDYVKKVNK
ATEAAMFLGWVEQLVYDFTDETSEVSTTDKIADITIIIPYIGPALNIGNMLYKDDFVGAL
IFSGAVILLEFIPEIAIPVLGTFALVSYIANKVLTVQTIDNALSKRNEKWDEVYKYIVTN
WLAKVNTQIDLIRKKMKEALENQAEATKAIINYQYNQYTEEEKNNINFNIDDLSSKLNES
INKAMININKFLNQCSVSYLMNSMIPYGVKRLEDFDASLKDALLKYIYDNRGTLIGQVDR
LKDKVNNTLSTDIPFQLSKYVDNQRLLSTFTEYIKNIINTSILNLRYESNHLIDLSRYAS
KINIGSKVNFDPIDKNQIQLFNLESSKIEVILKNAIVYNSMYENFSTSFWIRIPKYFNSI
SLNNEYTIINCMENNSGWKVSLNYGEIIWTLQDTQEIKQRVVFKYSQMINISDYINRWIF
VTITNNRLNNSKIYINGRLIDQKPISNLGNIHASNNIMFKLDGCRDTHRYIWIKYFNLFD
KELNEKEIKDLYDNQSNSGILKDFWGDYLQYDKPYYMLNLYDPNKYVDVNNVGIRGYMYL
KGPRGSVMTTNIYLNSSLYRGTKFIIKKYASGNKDNIVRNNDRVYINVVVKNKEYRLATN
ASQAGVEKILSALEIPDVGNLSQVVVMKSKNDQGITNKCKMNLQDNNGNDIGFIGFHQFN
NIAKLVASNWYNRQIERSSRTLGCSWEFIPVDDGWGERPLAATPAAPAPATPAAPAPTAP
AATPAAPAPATPAAPAPTAPAATPAAPAPATPAAPAPTAPAATPAAPAPATPAAPAPTAP
AATPAAPAPATPAAPAPTAPAGDLVPRGSANSSSVDKLWSHPQFEK
SEQ ID NO 8: (PA100-rBoNTA-PA100) (nucleic acid sequence)
ATGGGTAGCAGCCATCATCATCACCATCATGGTAGCCTGGTTCCGCGTAGCTCTTCTGCGGCGC
CGGCTGCCCCGGCGCCCGCCGCCCCTGCTGCGCCTGCGCCGGCAGCGCCAGCCGCGGCACC
GGCGGCTCCGGCGCCGGCCGCGCCCGCCGCACCGGCCCCAGCCGCGCCTGCAGCTGCACCG
GCGGCGCCGGCACCTGCGGCACCGGCGGCACCGGCCCCTGCGGCACCGGCCGCCGCGCCGG
CTGCACCTGCTCCGGCCGCGCCGGCGGCGCCAGCGCCGGCAGCGCCGGCAGCAGCACCGGC
GGCACCGGCGCCAGCTGCACCGGCGGCGCCAGCTCCTGCGGCGCCGGCCCCATTTGTGAACA
AGCAGTTTAACTATAAGGACCCGGTGAACGGTGTGGATATCGCGTATATCAAAATCCCGAATGCG
GGCCAGATGCAACCAGTCAAGGCGTTCAAGATTCATAACAAGATTTGGGTTATTCCGGAACGTGA
TACCTTCACCAATCCGGAAGAAGGCGATTTAAATCCGCCGCCAGAAGCCAAACAAGTGCCGGTG
AGCTACTATGATAGCACGTATCTTAGCACCGATAATGAAAAAGACAATTACCTGAAGGGCGTGAC
CAAGTTGTTCGAGCGCATCTACAGTACCGACTTAGGCCGCATGTTGTTGACGAGCATCGTTCGCG
GTATCCCGTTCTGGGGCGGCTCGACCATTGATACCGAGTTGAAAGTCATTGACACGAACTGTATC
AATGTTATCCAACCGGACGGCAGTTATCGCAGCGAGGAGTTAAATTTGGTCATCATCGGTCCAAG
CGCAGATATTATTCAGTTCGAATGCAAGAGCTTCGGCCATGAGGTCTTGAATTTGACGCGCAACG
GTTACGGCAGCACCCAATACATCCGCTTTAGCCCGGATTTCACCTTTGGCTTCGAGGAGAGCTTG
GAGGTGGACACCAACCCGCTGTTAGGTGCCGGCAAATTCGCAACCGACCCGGCAGTGACGTTG
GCGCACGAATTGATTCATGCGGGTCACCGCTTATACGGTATCGCGATCAATCCGAATCGCGTCTT
TAAAGTCAATACCAACGCGTACTACGAAATGAGCGGCTTAGAGGTTAGCTTTGAAGAATTACGCA
CCTTCGGTGGCCACGACGCCAAGTTCATCGACAGCCTGCAGGAAAATGAGTTCCGCTTGTACTAT
TACAATAAATTCAAGGACATCGCGAGCACCTTAAATAAAGCAAAGAGCATTGTGGGCACCACCGC
AAGCTTGCAGTACATGAAGAACGTATTTAAGGAAAAATATTTGTTGTCGGAGGATACCAGCGGGA
AATTCAGCGTCGATAAGCTGAAATTCGACAAATTGTATAAAATGCTGACCGAGATTTACACCGAGG
ATAACTTCGTCAAGTTTTTTAAGGTGTTAAATCGTAAGACCTATTTAAACTTTGATAAAGCGGTGTT
TAAAATTAATATCGTGCCGAAGGTGAATTACACCATCTACGATGGTTTCAATTTACGCAACACGAA
TCTGGCGGCGAATTTTAATGGCCAAAACACCGAAATTAACAACATGAACTTTACGAAGTTAAAGAA
TTTCACGGGCTTATTCGAATTCTACAAGTTATTATGCGTGCGCGGCATCATTACCAGCAAGGCAG
GTGCGGGCAAGTCCTTGGTTCCGCGTGGCAGCGCCGGCGCCGGCGCGCTCAATGATCTGTGTA
TTAAAGTCAATAACTGGGACCTGTTCTTCAGCCCGAGCGAGGATAACTTTACCAACGACTTAAACA
AAGGCGAGGAGATCACGAGCGATACGAACATCGAGGCGGCGGAGGAAAATATTAGCCTGGACC
TCATTCAGCAGTACTATCTGACGTTCAATTTTGACAATGAGCCGGAGAACATCAGCATTGAAAATC
TCAGCAGCGACATCATCGGTCAGTTGGAACTGATGCCGAACATTGAACGCTTTCCGAACGGCAAA
AAATATGAACTGGACAAGTATACCATGTTCCATTACTTACGCGCACAGGAATTTGAGCACGGCAA
GAGCCGCATTGCGCTGACCAATAGCGTTAACGAGGCCTTGTTAAATCCGAGCCGTGTCTACACG
TTCTTCAGCAGCGATTATGTCAAAAAAGTGAACAAGGCGACCGAAGCCGCGATGTTTTTGGGCTG
GGTCGAGCAATTGGTTTACGATTTTACCGACGAAACCAGCGAGGTGAGCACGACCGACAAAATT
GCAGATATCACCATCATCATTCCGTACATCGGTCCGGCGCTCAATATCGGCAATATGTTATACAA
GGACGACTTTGTGGGCGCGCTGATCTTTAGCGGCGCGGTTATCTTATTAGAATTCATCCCGGAGA
TCGCAATCCCGGTCTTGGGCACCTTTGCGTTGGTGAGCTATATCGCGAATAAAGTGCTCACGGTC
CAAACCATCGATAACGCGCTCAGCAAGCGTAATGAGAAATGGGACGAGGTTTATAAGTATATCGT
GACCAACTGGTTAGCAAAAGTCAATACGCAGATCGATCTCATCCGCAAAAAAATGAAAGAAGCCT
TGGAAAATCAAGCGGAGGCAACCAAAGCCATCATTAATTACCAGTATAACCAATATACCGAAGAA
GAAAAAAACAATATCAACTTCAATATCGATGATTTGAGCAGCAAACTGAACGAGAGCATTAACAAA
GCGATGATTAACATCAACAAGTTCTTGAATCAATGCAGCGTGAGCTATCTCATGAACAGCATGATC
CCGTATGGCGTCAAACGCTTGGAAGATTTTGACGCCAGCCTGAAAGATGCGCTCCTCAAGTATAT
TTATGACAACCGCGGCACCCTCATTGGCCAGGTGGACCGCTTGAAGGATAAAGTGAACAATACG
CTCAGCACGGATATCCCGTTCCAGCTGAGCAAGTACGTCGACAACCAGCGCTTACTGAGCACCT
TTACCGAGTATATCAAGAACATCATTAATACCAGCATCCTCAACTTGCGCTATGAGAGCAATCACC
TGATCGACCTCAGCCGCTACGCCAGCAAGATCAACATCGGCAGCAAGGTCAATTTCGACCCGAT
CGATAAGAATCAGATCCAATTGTTTAACCTGGAAAGCAGCAAGATCGAGGTTATCTTGAAGAACG
CGATTGTGTACAACAGCATGTACGAGAACTTTAGCACGAGCTTCTGGATTCGTATCCCGAAGTAT
TTCAATAGCATTAGCCTGAATAACGAATATACCATTATCAACTGCATGGAAAATAATAGCGGCTGG
AAGGTGAGCTTAAATTACGGCGAGATCATTTGGACCTTACAGGATACCCAAGAAATCAAACAGCG
CGTCGTCTTTAAGTATAGCCAGATGATCAACATCAGCGATTACATCAACCGCTGGATCTTCGTGA
CCATCACCAATAATCGCTTGAATAATAGCAAGATTTACATCAATGGTCGCTTGATTGATCAAAAAC
CGATCAGCAATCTCGGTAATATCCATGCCAGCAATAACATCATGTTTAAGTTAGACGGTTGCCGC
GATACCCACCGCTATATCTGGATCAAGTATTTTAACTTATTTGATAAGGAACTCAACGAAAAGGAA
ATTAAAGACTTATATGACAATCAGAGCAATAGCGGCATCCTGAAGGATTTCTGGGGCGACTACCT
GCAGTACGATAAGCCGTACTATATGTTGAACTTGTATGACCCGAACAAATATGTCGATGTGAACAA
TGTGGGTATTCGTGGCTATATGTACTTAAAGGGCCCGCGTGGTAGCGTGATGACCACGAATATTT
ACTTAAACAGCAGCTTATACCGCGGCACGAAGTTTATTATCAAGAAGTATGCCAGCGGCAACAAG
GACAATATCGTCCGCAACAACGACCGTGTGTATATTAACGTGGTGGTGAAGAATAAAGAGTACCG
CTTGGCCACGAATGCGAGCCAGGCGGGCGTGGAAAAAATCTTGAGCGCGTTGGAGATCCCGGA
CGTCGGCAACCTCAGCCAGGTTGTGGTGATGAAGTCTAAAAACGACCAGGGCATCACGAACAAG
TGCAAAATGAATTTGCAAGATAACAACGGCAACGACATCGGCTTTATTGGTTTTCACCAGTTCAAT
AACATCGCCAAACTCGTGGCCAGCAATTGGTATAACCGCCAAATTGAACGCAGCAGCCGCACGC
TCGGCTGTAGCTGGGAGTTCATCCCGGTGGACGATGGCTGGGGCGAGCGCCCGCTCGCGGCG
CCGGCTGCCCCGGCGCCCGCCGCCCCTGCTGCGCCTGCGCCGGCAGCGCCAGCCGCGGCACC
GGCGGCTCCGGCGCCGGCCGCGCCCGCCGCACCGGCCCCAGCCGCGCCTGCAGCTGCACCG
GCGGCGCCGGCACCTGCGGCACCGGCGGCACCGGCCCCTGCGGCACCGGCCGCCGCGCCGG
CTGCACCTGCTCCGGCCGCGCCGGCGGCGCCAGCGCCGGCAGCGCCGGCAGCAGCACCGGC
GGCACCGGCGCCAGCTGCACCGGCGGCGCCAGCTCCTGCGGCGCCGGCCGGAGATCTGGTGC
CACGCGGTTCCGCGAATTCGAGCTCCGTCGACAAGCTTTGGAGCCACCCGCAGTTCGAAAAATAA
SEQ ID NO 9: (PAY100-rBoNTA-PAY100) (nucleic acid sequence)
ATGGGTAGCAGCCATCATCATCACCATCATGGTAGCCTGGTTCCGCGTAGCTCTTCTGCGTACCC
GGCGGCGCCGGCGCCGGCGTACCCGGCGGCGCCGGCGCCGTACGCGCCGGCGGCGTACCCG
GCGGCGCCGGCGCCGGCGTACCCGGCGGCGCCGGCGCCGTACGCGCCGGCGGCGTACCCGG
CGGCGCCGGCGCCGGCGTACCCGGCGGCGCCGGCGCCGTACGCGCCGGCGGCGTACCCGGC
GGCGCCGGCGCCGGCGTACCCGGCGGCGCCGGCGCCGTACGCGCCGGCGGCGTACCCGGCG
GCGCCGGCGCCGGCGTACCCGGCGGCGCCGGCGCCGTACGCGCCGGCGCCATTTGTGAACAA
GCAGTTTAACTATAAGGACCCGGTGAACGGTGTGGATATCGCGTATATCAAAATCCCGAATGCGG
GCCAGATGCAACCAGTCAAGGCGTTCAAGATTCATAACAAGATTTGGGTTATTCCGGAACGTGAT
ACCTTCACCAATCCGGAAGAAGGCGATTTAAATCCGCCGCCAGAAGCCAAACAAGTGCCGGTGA
GCTACTATGATAGCACGTATCTTAGCACCGATAATGAAAAAGACAATTACCTGAAGGGCGTGACC
AAGTTGTTCGAGCGCATCTACAGTACCGACTTAGGCCGCATGTTGTTGACGAGCATCGTTCGCG
GTATCCCGTTCTGGGGCGGCTCGACCATTGATACCGAGTTGAAAGTCATTGACACGAACTGTATC
AATGTTATCCAACCGGACGGCAGTTATCGCAGCGAGGAGTTAAATTTGGTCATCATCGGTCCAAG
CGCAGATATTATTCAGTTCGAATGCAAGAGCTTCGGCCATGAGGTCTTGAATTTGACGCGCAACG
GTTACGGCAGCACCCAATACATCCGCTTTAGCCCGGATTTCACCTTTGGCTTCGAGGAGAGCTTG
GAGGTGGACACCAACCCGCTGTTAGGTGCCGGCAAATTCGCAACCGACCCGGCAGTGACGTTG
GCGCACGAATTGATTCATGCGGGTCACCGCTTATACGGTATCGCGATCAATCCGAATCGCGTCTT
TAAAGTCAATACCAACGCGTACTACGAAATGAGCGGCTTAGAGGTTAGCTTTGAAGAATTACGCA
CCTTCGGTGGCCACGACGCCAAGTTCATCGACAGCCTGCAGGAAAATGAGTTCCGCTTGTACTAT
TACAATAAATTCAAGGACATCGCGAGCACCTTAAATAAAGCAAAGAGCATTGTGGGCACCACCGC
AAGCTTGCAGTACATGAAGAACGTATTTAAGGAAAAATATTTGTTGTCGGAGGATACCAGCGGGA
AATTCAGCGTCGATAAGCTGAAATTCGACAAATTGTATAAAATGCTGACCGAGATTTACACCGAGG
ATAACTTCGTCAAGTTTTTTAAGGTGTTAAATCGTAAGACCTATTTAAACTTTGATAAAGCGGTGTT
TAAAATTAATATCGTGCCGAAGGTGAATTACACCATCTACGATGGTTTCAATTTACGCAACACGAA
TCTGGCGGCGAATTTTAATGGCCAAAACACCGAAATTAACAACATGAACTTTACGAAGTTAAAGAA
TTTCACGGGCTTATTCGAATTCTACAAGTTATTATGCGTGCGCGGCATCATTACCAGCAAGGCAG
GTGCGGGCAAGTCCTTGGTACCGCGTGGCAGCGCCGGCGCCGGCGCGCTCAATGATCTGTGTA
TTAAAGTCAATAACTGGGACCTGTTCTTCAGCCCGAGCGAGGATAACTTTACCAACGACTTAAACA
AAGGCGAGGAGATCACGAGCGATACGAACATCGAGGCGGCGGAGGAAAATATTAGCCTGGACC
TCATTCAGCAGTACTATCTGACGTTCAATTTTGACAATGAGCCGGAGAACATCAGCATTGAAAATC
TCAGCAGCGACATCATCGGTCAGTTGGAACTGATGCCGAACATTGAACGCTTTCCGAACGGCAAA
AAATATGAACTGGACAAGTATACCATGTTCCATTACTTACGCGCACAGGAATTTGAGCACGGCAA
GAGCCGCATTGCGCTGACCAATAGCGTTAACGAGGCCTTGTTAAATCCGAGCCGTGTCTACACG
TTCTTCAGCAGCGATTATGTCAAAAAAGTGAACAAGGCGACCGAAGCCGCGATGTTTTTGGGCTG
GGTCGAGCAATTGGTTTACGATTTTACCGACGAAACCAGCGAGGTGAGCACGACCGACAAAATT
GCAGATATCACCATCATCATTCCGTACATCGGTCCGGCGCTCAATATCGGCAATATGTTATACAA
GGACGACTTTGTGGGCGCGCTGATCTTTAGCGGCGCGGTTATCTTATTAGAATTCATCCCGGAGA
TCGCAATCCCGGTCTTGGGCACCTTTGCGTTGGTGAGCTATATCGCGAATAAAGTGCTCACGGTC
CAAACCATCGATAACGCGCTCAGCAAGCGTAATGAGAAATGGGACGAGGTTTATAAGTATATCGT
GACCAACTGGTTAGCAAAAGTCAATACGCAGATCGATCTCATCCGCAAAAAAATGAAAGAAGCCT
TGGAAAATCAAGCGGAGGCAACCAAAGCCATCATTAATTACCAGTATAACCAATATACCGAAGAA
GAAAAAAACAATATCAACTTCAATATCGATGATTTGAGCAGCAAACTGAACGAGAGCATTAACAAA
GCGATGATTAACATCAACAAGTTCTTGAATCAATGCAGCGTGAGCTATCTCATGAACAGCATGATC
CCGTATGGCGTCAAACGCTTGGAAGATTTTGACGCCAGCCTGAAAGATGCGCTCCTCAAGTATAT
TTATGACAACCGCGGCACCCTCATTGGCCAGGTGGACCGCTTGAAGGATAAAGTGAACAATACG
CTCAGCACGGATATCCCGTTCCAGCTGAGCAAGTACGTCGACAACCAGCGCTTACTGAGCACCT
TTACCGAGTATATCAAGAACATCATTAATACCAGCATCCTCAACTTGCGCTATGAGAGCAATCACC
TGATCGACCTCAGCCGCTACGCCAGCAAGATCAACATCGGCAGCAAGGTCAATTTCGACCCGAT
CGATAAGAATCAGATCCAATTGTTTAACCTGGAAAGCAGCAAGATCGAGGTTATCTTGAAGAACG
CGATTGTGTACAACAGCATGTACGAGAACTTTAGCACGAGCTTCTGGATTCGTATCCCGAAGTAT
TTCAATAGCATTAGCCTGAATAACGAATATACCATTATCAACTGCATGGAAAATAATAGCGGCTGG
AAGGTGAGCTTAAATTACGGCGAGATCATTTGGACCTTACAGGATACCCAAGAAATCAAACAGCG
CGTCGTCTTTAAGTATAGCCAGATGATCAACATCAGCGATTACATCAACCGCTGGATCTTCGTGA
CCATCACCAATAATCGCTTGAATAATAGCAAGATTTACATCAATGGTCGCTTGATTGATCAAAAAC
CGATCAGCAATCTCGGTAATATCCATGCCAGCAATAACATCATGTTTAAGTTAGACGGTTGCCGC
GATACCCACCGCTATATCTGGATCAAGTATTTTAACTTATTTGATAAGGAACTCAACGAAAAGGAA
ATTAAAGACTTATATGACAATCAGAGCAATAGCGGCATCCTGAAGGATTTCTGGGGCGACTACCT
GCAGTACGATAAGCCGTACTATATGTTGAACTTGTATGACCCGAACAAATATGTCGATGTGAACAA
TGTGGGTATTCGTGGCTATATGTACTTAAAGGGCCCGCGTGGTAGCGTGATGACCACGAATATTT
ACTTAAACAGCAGCTTATACCGCGGCACGAAGTTTATTATCAAGAAGTATGCCAGCGGCAACAAG
GACAATATCGTCCGCAACAACGACCGTGTGTATATTAACGTGGTGGTGAAGAATAAAGAGTACCG
CTTGGCCACGAATGCGAGCCAGGCGGGCGTGGAAAAAATCTTGAGCGCGTTGGAGATCCCGGA
CGTCGGCAACCTCAGCCAGGTTGTGGTGATGAAGTCTAAAAACGACCAGGGCATCACGAACAAG
TGCAAAATGAATTTGCAAGATAACAACGGCAACGACATCGGCTTTATTGGTTTTCACCAGTTCAAT
AACATCGCCAAACTCGTGGCCAGCAATTGGTATAACCGCCAAATTGAACGCAGCAGCCGCACGC
TCGGCTGTAGCTGGGAGTTCATCCCGGTGGACGATGGCTGGGGCGAGCGCCCGCTCGCGGCGT
ACCCGGCGGCGCCGGCGCCGGCGTACCCGGCGGCGCCGGCGCCGTACGCGCCGGCGGCGTA
CCCGGCGGCGCCGGCGCCGGCGTACCCGGCGGCGCCGGCGCCGTACGCGCCGGCGGCGTAC
CCGGCGGCGCCGGCGCCGGCGTACCCGGCGGCGCCGGCGCCGTACGCGCCGGCGGCGTACC
CGGCGGCGCCGGCGCCGGCGTACCCGGCGGCGCCGGCGCCGTACGCGCCGGCGGCGTACCC
GGCGGCGCCGGCGCCGGCGTACCCGGCGGCGCCGGCGCCGTACGCGCCGGCGGGAGATCTG
GTGCCACGCGGTTCCGCGAATTCGAGCTCCGTCGACAAGCTTTGGAGCCACCCGCAGTTCGAAA
AATAA
SEQ ID NO 10: (PAQ100-rBoNTA-PAQ100) (nucleic acid sequence)
ATGGGTAGCAGCCATCATCATCACCATCATGGTAGCCTGGTTCCGCGTAGCTCTTCTGCGCAGC
CGGCGGCGCCGGCGCCGGCGCAGCCGGCGGCGCCGGCGCCGCAGGCGCCGGCGGCGCAGC
CGGCGGCGCCGGCGCCGGCGCAGCCGGCGGCGCCGGCGCCGCAGGCGCCGGCGGCGCAGC
CGGCGGCGCCGGCGCCGGCGCAGCCGGCGGCGCCGGCGCCGCAGGCGCCGGCGGCGCAGC
CGGCGGCGCCGGCGCCGGCGCAGCCGGCGGCGCCGGCGCCGCAGGCGCCGGCGGCGCAGC
CGGCGGCGCCGGCGCCGGCGCAGCCGGCGGCGCCGGCGCCGCAGGCGCCGGCGCCATTTGT
GAACAAGCAGTTTAACTATAAGGACCCGGTGAACGGTGTGGATATCGCGTATATCAAAATCCCGA
ATGCGGGCCAGATGCAACCAGTCAAGGCGTTCAAGATTCATAACAAGATTTGGGTTATTCCGGAA
CGTGATACCTTCACCAATCCGGAAGAAGGCGATTTAAATCCGCCGCCAGAAGCCAAACAAGTGC
CGGTGAGCTACTATGATAGCACGTATCTTAGCACCGATAATGAAAAAGACAATTACCTGAAGGGC
GTGACCAAGTTGTTCGAGCGCATCTACAGTACCGACTTAGGCCGCATGTTGTTGACGAGCATCGT
TCGCGGTATCCCGTTCTGGGGCGGCTCGACCATTGATACCGAGTTGAAAGTCATTGACACGAAC
TGTATCAATGTTATCCAACCGGACGGCAGTTATCGCAGCGAGGAGTTAAATTTGGTCATCATCGG
TCCAAGCGCAGATATTATTCAGTTCGAATGCAAGAGCTTCGGCCATGAGGTCTTGAATTTGACGC
GCAACGGTTACGGCAGCACCCAATACATCCGCTTTAGCCCGGATTTCACCTTTGGCTTCGAGGA
GAGCTTGGAGGTGGACACCAACCCGCTGTTAGGTGCCGGCAAATTCGCAACCGACCCGGCAGT
GACGTTGGCGCACGAATTGATTCATGCGGGTCACCGCTTATACGGTATCGCGATCAATCCGAATC
GCGTCTTTAAAGTCAATACCAACGCGTACTACGAAATGAGCGGCTTAGAGGTTAGCTTTGAAGAA
TTACGCACCTTCGGTGGCCACGACGCCAAGTTCATCGACAGCCTGCAGGAAAATGAGTTCCGCT
TGTACTATTACAATAAATTCAAGGACATCGCGAGCACCTTAAATAAAGCAAAGAGCATTGTGGGCA
CCACCGCAAGCTTGCAGTACATGAAGAACGTATTTAAGGAAAAATATTTGTTGTCGGAGGATACC
AGCGGGAAATTCAGCGTCGATAAGCTGAAATTCGACAAATTGTATAAAATGCTGACCGAGATTTA
CACCGAGGATAACTTCGTCAAGTTTTTTAAGGTGTTAAATCGTAAGACCTATTTAAACTTTGATAAA
GCGGTGTTTAAAATTAATATCGTGCCGAAGGTGAATTACACCATCTACGATGGTTTCAATTTACGC
AACACGAATCTGGCGGCGAATTTTAATGGCCAAAACACCGAAATTAACAACATGAACTTTACGAA
GTTAAAGAATTTCACGGGCTTATTCGAATTCTACAAGTTATTATGCGTGCGCGGCATCATTACCAG
CAAGGCAGGTGCGGGCAAGTCCTTGGTACCGCGTGGCAGCGCCGGCGCCGGCGCGCTCAATG
ATCTGTGTATTAAAGTCAATAACTGGGACCTGTTCTTCAGCCCGAGCGAGGATAACTTTACCAAC
GACTTAAACAAAGGCGAGGAGATCACGAGCGATACGAACATCGAGGCGGCGGAGGAAAATATTA
GCCTGGACCTCATTCAGCAGTACTATCTGACGTTCAATTTTGACAATGAGCCGGAGAACATCAGC
ATTGAAAATCTCAGCAGCGACATCATCGGTCAGTTGGAACTGATGCCGAACATTGAACGCTTTCC
GAACGGCAAAAAATATGAACTGGACAAGTATACCATGTTCCATTACTTACGCGCACAGGAATTTGA
GCACGGCAAGAGCCGCATTGCGCTGACCAATAGCGTTAACGAGGCCTTGTTAAATCCGAGCCGT
GTCTACACGTTCTTCAGCAGCGATTATGTCAAAAAAGTGAACAAGGCGACCGAAGCCGCGATGTT
TTTGGGCTGGGTCGAGCAATTGGTTTACGATTTTACCGACGAAACCAGCGAGGTGAGCACGACC
GACAAAATTGCAGATATCACCATCATCATTCCGTACATCGGTCCGGCGCTCAATATCGGCAATAT
GTTATACAAGGACGACTTTGTGGGCGCGCTGATCTTTAGCGGCGCGGTTATCTTATTAGAATTCA
TCCCGGAGATCGCAATCCCGGTCTTGGGCACCTTTGCGTTGGTGAGCTATATCGCGAATAAAGT
GCTCACGGTCCAAACCATCGATAACGCGCTCAGCAAGCGTAATGAGAAATGGGACGAGGTTTAT
AAGTATATCGTGACCAACTGGTTAGCAAAAGTCAATACGCAGATCGATCTCATCCGCAAAAAAATG
AAAGAAGCCTTGGAAAATCAAGCGGAGGCAACCAAAGCCATCATTAATTACCAGTATAACCAATAT
ACCGAAGAAGAAAAAAACAATATCAACTTCAATATCGATGATTTGAGCAGCAAACTGAACGAGAG
CATTAACAAAGCGATGATTAACATCAACAAGTTCTTGAATCAATGCAGCGTGAGCTATCTCATGAA
CAGCATGATCCCGTATGGCGTCAAACGCTTGGAAGATTTTGACGCCAGCCTGAAAGATGCGCTC
CTCAAGTATATTTATGACAACCGCGGCACCCTCATTGGCCAGGTGGACCGCTTGAAGGATAAAGT
GAACAATACGCTCAGCACGGATATCCCGTTCCAGCTGAGCAAGTACGTCGACAACCAGCGCTTA
CTGAGCACCTTTACCGAGTATATCAAGAACATCATTAATACCAGCATCCTCAACTTGCGCTATGAG
AGCAATCACCTGATCGACCTCAGCCGCTACGCCAGCAAGATCAACATCGGCAGCAAGGTCAATTT
CGACCCGATCGATAAGAATCAGATCCAATTGTTTAACCTGGAAAGCAGCAAGATCGAGGTTATCT
TGAAGAACGCGATTGTGTACAACAGCATGTACGAGAACTTTAGCACGAGCTTCTGGATTCGTATC
CCGAAGTATTTCAATAGCATTAGCCTGAATAACGAATATACCATTATCAACTGCATGGAAAATAATA
GCGGCTGGAAGGTGAGCTTAAATTACGGCGAGATCATTTGGACCTTACAGGATACCCAAGAAATC
AAACAGCGCGTCGTCTTTAAGTATAGCCAGATGATCAACATCAGCGATTACATCAACCGCTGGAT
CTTCGTGACCATCACCAATAATCGCTTGAATAATAGCAAGATTTACATCAATGGTCGCTTGATTGA
TCAAAAACCGATCAGCAATCTCGGTAATATCCATGCCAGCAATAACATCATGTTTAAGTTAGACGG
TTGCCGCGATACCCACCGCTATATCTGGATCAAGTATTTTAACTTATTTGATAAGGAACTCAACGA
AAAGGAAATTAAAGACTTATATGACAATCAGAGCAATAGCGGCATCCTGAAGGATTTCTGGGGCG
ACTACCTGCAGTACGATAAGCCGTACTATATGTTGAACTTGTATGACCCGAACAAATATGTCGATG
TGAACAATGTGGGTATTCGTGGCTATATGTACTTAAAGGGCCCGCGTGGTAGCGTGATGACCAC
GAATATTTACTTAAACAGCAGCTTATACCGCGGCACGAAGTTTATTATCAAGAAGTATGCCAGCGG
CAACAAGGACAATATCGTCCGCAACAACGACCGTGTGTATATTAACGTGGTGGTGAAGAATAAAG
AGTACCGCTTGGCCACGAATGCGAGCCAGGCGGGCGTGGAAAAAATCTTGAGCGCGTTGGAGA
TCCCGGACGTCGGCAACCTCAGCCAGGTTGTGGTGATGAAGTCTAAAAACGACCAGGGCATCAC
GAACAAGTGCAAAATGAATTTGCAAGATAACAACGGCAACGACATCGGCTTTATTGGTTTTCACCA
GTTCAATAACATCGCCAAACTCGTGGCCAGCAATTGGTATAACCGCCAAATTGAACGCAGCAGCC
GCACGCTCGGCTGTAGCTGGGAGTTCATCCCGGTGGACGATGGCTGGGGCGAGCGCCCGCTCG
CGGCGCAGCCGGCGGCGCCGGCGCCGGCGCAGCCGGCGGCGCCGGCGCCGCAGGCGCCGG
CGGCGCAGCCGGCGGCGCCGGCGCCGGCGCAGCCGGCGGCGCCGGCGCCGCAGGCGCCGG
CGGCGCAGCCGGCGGCGCCGGCGCCGGCGCAGCCGGCGGCGCCGGCGCCGCAGGCGCCGG
CGGCGCAGCCGGCGGCGCCGGCGCCGGCGCAGCCGGCGGCGCCGGCGCCGCAGGCGCCGG
CGGCGCAGCCGGCGGCGCCGGCGCCGGCGCAGCCGGCGGCGCCGGCGCCGCAGGCGCCGG
CGGGAGATCTGGTGCCACGCGGTTCCGCGAATTCGAGCTCCGTCGACAAGCTTTGGAGCCACC
CGCAGTTCGAAAAATAA
SEQ ID NO 11: (PAT100-rBoNTA-PAT100) (nucleic acid sequence)
ATGGGTAGCAGCCATCATCATCACCATCATGGTAGCCTGGTTCCGCGTAGCTCTTCTGCG
ACCCCGGCGGCGCCGGCGCCGGCGACCCCGGCGGCGCCGGCGCCGACCGCGCCGGCGGCG
ACCCCGGCGGCGCCGGCGCCGGCGACCCCGGCGGCGCCGGCGCCGACCGCGCCGGCGGCG
ACCCCGGCGGCGCCGGCGCCGGCGACCCCGGCGGCGCCGGCGCCGACCGCGCCGGCGGCG
ACCCCGGCGGCGCCGGCGCCGGCGACCCCGGCGGCGCCGGCGCCGACCGCGCCGGCGGCG
ACCCCGGCGGCGCCGGCGCCGGCGACCCCGGCGGCGCCGGCGCCGACCGCGCCGGCGCCA
TTTGTGAACAAGCAGTTTAACTATAAGGACCCGGTGAACGGTGTGGATATCGCGTATATC
AAAATCCCGAATGCGGGCCAGATGCAACCAGTCAAGGCGTTCAAGATTCATAACAAGATT
TGGGTTATTCCGGAACGTGATACCTTCACCAATCCGGAAGAAGGCGATTTAAATCCGCCG
CCAGAAGCCAAACAAGTGCCGGTGAGCTACTATGATAGCACGTATCTTAGCACCGATAAT
GAAAAAGACAATTACCTGAAGGGCGTGACCAAGTTGTTCGAGCGCATCTACAGTACCGAC
TTAGGCCGCATGTTGTTGACGAGCATCGTTCGCGGTATCCCGTTCTGGGGCGGCTCGACC
ATTGATACCGAGTTGAAAGTCATTGACACGAACTGTATCAATGTTATCCAACCGGACGGC
AGTTATCGCAGCGAGGAGTTAAATTTGGTCATCATCGGTCCAAGCGCAGATATTATTCAG
TTCGAATGCAAGAGCTTCGGCCATGAGGTCTTGAATTTGACGCGCAACGGTTACGGCAGC
ACCCAATACATCCGCTTTAGCCCGGATTTCACCTTTGGCTTCGAGGAGAGCTTGGAGGTG
GACACCAACCCGCTGTTAGGTGCCGGCAAATTCGCAACCGACCCGGCAGTGACGTTGGCG
CACGAATTGATTCATGCGGGTCACCGCTTATACGGTATCGCGATCAATCCGAATCGCGTC
TTTAAAGTCAATACCAACGCGTACTACGAAATGAGCGGCTTAGAGGTTAGCTTTGAAGAA
TTACGCACCTTCGGTGGCCACGACGCCAAGTTCATCGACAGCCTGCAGGAAAATGAGTTC
CGCTTGTACTATTACAATAAATTCAAGGACATCGCGAGCACCTTAAATAAAGCAAAGAGC
ATTGTGGGCACCACCGCAAGCTTGCAGTACATGAAGAACGTATTTAAGGAAAAATATTTG
TTGTCGGAGGATACCAGCGGGAAATTCAGCGTCGATAAGCTGAAATTCGACAAATTGTAT
AAAATGCTGACCGAGATTTACACCGAGGATAACTTCGTCAAGTTTTTTAAGGTGTTAAAT
CGTAAGACCTATTTAAACTTTGATAAAGCGGTGTTTAAAATTAATATCGTGCCGAAGGTG
AATTACACCATCTACGATGGTTTCAATTTACGCAACACGAATCTGGCGGCGAATTTTAAT
GGCCAAAACACCGAAATTAACAACATGAACTTTACGAAGTTAAAGAATTTCACGGGCTTA
TTCGAATTCTACAAGTTATTATGCGTGCGCGGCATCATTACCAGCAAGGCAGGTGCGGGC
AAGTCCTTGGTACCGCGTGGCAGCGCCGGCGCCGGCGCGCTCAATGATCTGTGTATTAAA
GTCAATAACTGGGACCTGTTCTTCAGCCCGAGCGAGGATAACTTTACCAACGACTTAAAC
AAAGGCGAGGAGATCACGAGCGATACGAACATCGAGGCGGCGGAGGAAAATATTAGCCTG
GACCTCATTCAGCAGTACTATCTGACGTTCAATTTTGACAATGAGCCGGAGAACATCAGC
ATTGAAAATCTCAGCAGCGACATCATCGGTCAGTTGGAACTGATGCCGAACATTGAACGC
TTTCCGAACGGCAAAAAATATGAACTGGACAAGTATACCATGTTCCATTACTTACGCGCA
CAGGAATTTGAGCACGGCAAGAGCCGCATTGCGCTGACCAATAGCGTTAACGAGGCCTTG
TTAAATCCGAGCCGTGTCTACACGTTCTTCAGCAGCGATTATGTCAAAAAAGTGAACAAG
GCGACCGAAGCCGCGATGTTTTTGGGCTGGGTCGAGCAATTGGTTTACGATTTTACCGAC
GAAACCAGCGAGGTGAGCACGACCGACAAAATTGCAGATATCACCATCATCATTCCGTAC
ATCGGTCCGGCGCTCAATATCGGCAATATGTTATACAAGGACGACTTTGTGGGCGCGCTG
ATCTTTAGCGGCGCGGTTATCTTATTAGAATTCATCCCGGAGATCGCAATCCCGGTCTTG
GGCACCTTTGCGTTGGTGAGCTATATCGCGAATAAAGTGCTCACGGTCCAAACCATCGAT
AACGCGCTCAGCAAGCGTAATGAGAAATGGGACGAGGTTTATAAGTATATCGTGACCAAC
TGGTTAGCAAAAGTCAATACGCAGATCGATCTCATCCGCAAAAAAATGAAAGAAGCCTTG
GAAAATCAAGCGGAGGCAACCAAAGCCATCATTAATTACCAGTATAACCAATATACCGAA
GAAGAAAAAAACAATATCAACTTCAATATCGATGATTTGAGCAGCAAACTGAACGAGAGC
ATTAACAAAGCGATGATTAACATCAACAAGTTCTTGAATCAATGCAGCGTGAGCTATCTC
ATGAACAGCATGATCCCGTATGGCGTCAAACGCTTGGAAGATTTTGACGCCAGCCTGAAA
GATGCGCTCCTCAAGTATATTTATGACAACCGCGGCACCCTCATTGGCCAGGTGGACCGC
TTGAAGGATAAAGTGAACAATACGCTCAGCACGGATATCCCGTTCCAGCTGAGCAAGTAC
GTCGACAACCAGCGCTTACTGAGCACCTTTACCGAGTATATCAAGAACATCATTAATACC
AGCATCCTCAACTTGCGCTATGAGAGCAATCACCTGATCGACCTCAGCCGCTACGCCAGC
AAGATCAACATCGGCAGCAAGGTCAATTTCGACCCGATCGATAAGAATCAGATCCAATTG
TTTAACCTGGAAAGCAGCAAGATCGAGGTTATCTTGAAGAACGCGATTGTGTACAACAGC
ATGTACGAGAACTTTAGCACGAGCTTCTGGATTCGTATCCCGAAGTATTTCAATAGCATT
AGCCTGAATAACGAATATACCATTATCAACTGCATGGAAAATAATAGCGGCTGGAAGGTG
AGCTTAAATTACGGCGAGATCATTTGGACCTTACAGGATACCCAAGAAATCAAACAGCGC
GTCGTCTTTAAGTATAGCCAGATGATCAACATCAGCGATTACATCAACCGCTGGATCTTC
GTGACCATCACCAATAATCGCTTGAATAATAGCAAGATTTACATCAATGGTCGCTTGATT
GATCAAAAACCGATCAGCAATCTCGGTAATATCCATGCCAGCAATAACATCATGTTTAAG
TTAGACGGTTGCCGCGATACCCACCGCTATATCTGGATCAAGTATTTTAACTTATTTGAT
AAGGAACTCAACGAAAAGGAAATTAAAGACTTATATGACAATCAGAGCAATAGCGGCATC
CTGAAGGATTTCTGGGGCGACTACCTGCAGTACGATAAGCCGTACTATATGTTGAACTTG
TATGACCCGAACAAATATGTCGATGTGAACAATGTGGGTATTCGTGGCTATATGTACTTA
AAGGGCCCGCGTGGTAGCGTGATGACCACGAATATTTACTTAAACAGCAGCTTATACCGC
GGCACGAAGTTTATTATCAAGAAGTATGCCAGCGGCAACAAGGACAATATCGTCCGCAAC
AACGACCGTGTGTATATTAACGTGGTGGTGAAGAATAAAGAGTACCGCTTGGCCACGAAT
GCGAGCCAGGCGGGCGTGGAAAAAATCTTGAGCGCGTTGGAGATCCCGGACGTCGGCAAC
CTCAGCCAGGTTGTGGTGATGAAGTCTAAAAACGACCAGGGCATCACGAACAAGTGCAAA
ATGAATTTGCAAGATAACAACGGCAACGACATCGGCTTTATTGGTTTTCACCAGTTCAAT
AACATCGCCAAACTCGTGGCCAGCAATTGGTATAACCGCCAAATTGAACGCAGCAGCCGC
ACGCTCGGCTGTAGCTGGGAGTTCATCCCGGTGGACGATGGCTGGGGCGAGCGCCCGCTC
GCGGCGACCCCGGCGGCGCCGGCGCCGGCGACCCCGGCGGCGCCGGCGCCGACCGCGCCG
GCGGCGACCCCGGCGGCGCCGGCGCCGGCGACCCCGGCGGCGCCGGCGCCGACCGCGCCG
GCGGCGACCCCGGCGGCGCCGGCGCCGGCGACCCCGGCGGCGCCGGCGCCGACCGCGCCG
GCGGCGACCCCGGCGGCGCCGGCGCCGGCGACCCCGGCGGCGCCGGCGCCGACCGCGCCG
GCGGCGACCCCGGCGGCGCCGGCGCCGGCGACCCCGGCGGCGCCGGCGCCGACCGCGCCG
GCGGGAGATCTGGTGCCACGCGGTTCCGCGAATTCGAGCTCCGTCGACAAGCTTTGGAGC
CACCCGCAGTTCGAAAAATAA

1. A recombinant clostridial neurotoxin comprising at least one domain wherein said domain comprises an amino acid sequence consisting of at least 50 amino acid residues, wherein said amino acid sequence consists of at least one proline and at least one alanine residue, or wherein said domain comprises a plurality of amino acid repeats, wherein said repeats consist of proline and alanine residues and wherein no more than six consecutive amino acid residues are identical.

2. The recombinant clostridial neurotoxin of claim 1, wherein said domain comprises a plurality of amino acid repeats consisting of (AAPAA PAPAA PAAPA PAAPA)n with n being an integer selected from 3 to 25, in particular wherein n is 5.

3. A recombinant clostridial neurotoxin comprising at least one domain wherein said domain comprises an amino acid sequence consisting of at least 50 amino acid residues, wherein said domain comprises a plurality of amino acid repeats, wherein said repeats consist of an amino acid sequence selected from the group consisting of
(SEQ ID NO: 1)
AYPAAPAPAYPAAPAPYAPA,
(SEQ ID NO: 2)
AQPAAPAPAQPAAPAPQAPA
and
(SEQ ID NO: 3)
ATPAAPAPATPAAPAPTAPA.

4. The recombinant clostridial neurotoxin of claim 1, wherein said at least one domain comprises an amino acid sequence consisting of between 50 and 500 amino acid residues, more particularly between 70 and 300 amino acid residues, particularly 100 amino acid residues, 150 amino acid residues, or 200 amino acid residues.

5. The recombinant clostridial neurotoxin of claim 1, wherein the sequence of said clostridial neurotoxin is selected from the sequence of (i) a Clostridium botulinum neurotoxin serotype A, B, C, D, E, F, and G, or (ii) from the sequence of a functional variant of a Clostridium botulinum neurotoxin of (i).

6. The recombinant clostridial neurotoxin claim 1, wherein said at least one domain is inserted at a position selected from (i) the N-terminus of the light chain of said recombinant clostridial neurotoxin; (ii) the C-terminus of the light chain of said recombinant clostridial neurotoxin; (iii) the N-terminus of the heavy chain of said recombinant clostridial neurotoxin; or (iv) the C-terminus of the heavy chain of said recombinant clostridial neurotoxin.

7. The recombinant clostridial neurotoxin of claim 1, wherein the neurotoxin comprises two of said domains, wherein both domains comprise an amino acid sequence consisting of at least one proline and at least one alanine residue.

8. The recombinant clostridial neurotoxin of claim 3, wherein the neurotoxin comprises two of said domains, wherein both domains comprise a plurality of amino acid repeats selected from the group consisting of
(SEQ ID NO: 1)
AYPAAPAPAYPAAPAPYAPA,
(SEQ ID NO: 2)
AQPAAPAPAQPAAPAPQAPA
and
(SEQ ID NO: 3)
ATPAAPAPATPAAPAPTAPA.

9. The recombinant clostridial neurotoxin of claim 7, wherein one domain is inserted at a position of the N-terminus of the light chain of said recombinant clostridial neurotoxin and the second domain is inserted at a position of the C-terminus of the heavy chain of said recombinant clostridial neurotoxin.

10. The recombinant clostridial neurotoxin of claim 1, wherein said recombinant clostridial neurotoxin shows an increased duration of effect relative to an identical clostridial neurotoxin without said domain(s).

11. A composition comprising the recombinant clostridial neurotoxin of claim 1.

12. A pharmaceutical composition comprising the recombinant clostridial neurotoxin of claim 1.

13. Use of the recombinant clostridial neurotoxin of claim 1 for cosmetic treatment.

14. A method for the generation of a recombinant clostridial neurotoxin according to claim 1, comprising the step of obtaining a recombinant nucleic acid sequence encoding a recombinant single-chain precursor clostridial neurotoxin by the insertion of a nucleic acid sequence encoding said domain into a nucleic acid sequence encoding a parental clostridial neurotoxin, or wherein said method further comprises the step of heterologously expressing said recombinant nucleic acid sequence in a host cell, particularly in a bacterial host cell, more particularly in an E. coli host cell.

15. A recombinant single-chain clostridial neurotoxin, which is a precursor for the recombinant clostridial neurotoxin of claim 1.


SEQUENCE LISTING (11 sequences)

SEQ ID NO: 1 - protein, 100 aa, Artificial Sequence
  PAY100
AYPAAPAPAYPAAPAPYAPAAYPAAPAPAYPAAPAPYAPAAYPAAPAPAYPAAPAPYAPAAYPAAPAPAYPAAPAPYAPAAYPAAPAPAYPAAPAPYAPA

SEQ ID NO: 2 - protein, 100 aa, Artificial Sequence
  PAQ100
AQPAAPAPAQPAAPAPQAPAAQPAAPAPAQPAAPAPQAPAAQPAAPAPAQPAAPAPQAPAAQPAAPAPAQPAAPAPQAPAAQPAAPAPAQPAAPAPQAPA

SEQ ID NO: 3 - protein, 100 aa, Artificial Sequence
  PAT100
ATPAAPAPATPAAPAPTAPAATPAAPAPATPAAPAPTAPAATPAAPAPATPAAPAPTAPAATPAAPAPATPAAPAPTAPAATPAAPAPATPAAPAPTAPA

SEQ ID NO: 4 - protein, 1545 aa, Artificial Sequence
  PA100-rBoNTA-PA100
MGSSHHHHHHGSLVPRSSSAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAGDLVPRGSANSSSVDKLWSHPQFEK

SEQ ID NO: 5 - protein, 1546 aa, Artificial Sequence
  PAY100-rBoNTA-PAY100
MGSSHHHHHHGSLVPRSSSAYPAAPAPAYPAAPAPYAPAAYPAAPAPAYPAAPAPYAPAAYPAAPAPAYPAAPAPYAPAAYPAAPAPAYPAAPAPYAPAAYPAAPAPAYPAAPAPYAPAPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLAAYPAAPAPAYPAAPAPYAPAAYPAAPAPAYPAAPAPYAPAAYPAAPAPAYPAAPAPYAPAAYPAAPAPAYPAAPAPYAPAAYPAAPAPAYPAAPAPQAPAGDLVPRGSANSSSVDKLWSHPQFEK

SEQ ID NO: 6 - protein, 1546 aa, Artificial Sequence
  PAQ100-rBoNTA-PAQ100
MGSSHHHHHHGSLVPRSSSAQPAAPAPAQPAAPAPQAPAAQPAAPAPAQPAAPAPQAPAAQPAAPAPAQPAAPAPQAPAAQPAAPAPAQPAAPAPQAPAAQPAAPAPAQPAAPAPQAPAPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLAAQPAAPAPAQPAAPAPQAPAAQPAAPAPAQPAAPAPQAPAAQPAAPAPAQPAAPAPQAPAAQPAAPAPAQPAAPAPQAPAAQPAAPAPAQPAAPAPQAPAGDLVPRGSANSSSVDKLWSHPQFEK

SEQ ID NO: 7 - protein, 1546 aa, Artificial Sequence
  PAT100-rBoNTA-PAT100
MGSSHHHHHHGSLVPRSSSATPAAPAPATPAAPAPTAPAATPAAPAPATPAAPAPTAPAATPAAPAPATPAAPAPTAPAATPAAPAPATPAAPAPTAPAATPAAPAPATPAAPAPTAPAPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLAATPAAPAPATPAAPAPTAPAATPAAPAPATPAAPAPTAPAATPAAPAPATPAAPAPTAPAATPAAPAPATPAAPAPTAPAATPAAPAPATPAAPAPTAPAGDLVPRGSANSSSVDKLWSHPQFEK

SEQ ID NO: 8 - DNA, 4638 bp, Artificial Sequence
  PA100-rBoNTA-PA100
atgggtagcagccatcatcatcaccatcatggtagcctggttccgcgtagctcttctgcggcgccggctgccccggcgcccgccgcccctgctgcgcctgcgccggcagcgccagccgcggcaccggcggctccggcgccggccgcgcccgccgcaccggccccagccgcgcctgcagctgcaccggcggcgccggcacctgcggcaccggcggcaccggcccctgcggcaccggccgccgcgccggctgcacctgctccggccgcgccggcggcgccagcgccggcagcgccggcagcagcaccggcggcaccggcgccagctgcaccggcggcgccagctcctgcggcgccggccccatttgtgaacaagcagtttaactataaggacccggtgaacggtgtggatatcgcgtatatcaaaatcccgaatgcgggccagatgcaaccagtcaaggcgttcaagattcataacaagatttgggttattccggaacgtgataccttcaccaatccggaagaaggcgatttaaatccgccgccagaagccaaacaagtgccggtgagctactatgatagcacgtatcttagcaccgataatgaaaaagacaattacctgaagggcgtgaccaagttgttcgagcgcatctacagtaccgacttaggccgcatgttgttgacgagcatcgttcgcggtatcccgttctggggcggctcgaccattgataccgagttgaaagtcattgacacgaactgtatcaatgttatccaaccggacggcagttatcgcagcgaggagttaaatttggtcatcatcggtccaagcgcagatattattcagttcgaatgcaagagcttcggccatgaggtcttgaatttgacgcgcaacggttacggcagcacccaatacatccgctttagcccggatttcacctttggcttcgaggagagcttggaggtggacaccaacccgctgttaggtgccggcaaattcgcaaccgacccggcagtgacgttggcgcacgaattgattcatgcgggtcaccgcttatacggtatcgcgatcaatccgaatcgcgtctttaaagtcaataccaacgcgtactacgaaatgagcggcttagaggttagctttgaagaattacgcaccttcggtggccacgacgccaagttcatcgacagcctgcaggaaaatgagttccgcttgtactattacaataaattcaaggacatcgcgagcaccttaaataaagcaaagagcattgtgggcaccaccgcaagcttgcagtacatgaagaacgtatttaaggaaaaatatttgttgtcggaggataccagcgggaaattcagcgtcgataagctgaaattcgacaaattgtataaaatgctgaccgagatttacaccgaggataacttcgtcaagttttttaaggtgttaaatcgtaagacctatttaaactttgataaagcggtgtttaaaattaatatcgtgccgaaggtgaattacaccatctacgatggtttcaatttacgcaacacgaatctggcggcgaattttaatggccaaaacaccgaaattaacaacatgaactttacgaagttaaagaatttcacgggcttattcgaattctacaagttattatgcgtgcgcggcatcattaccagcaaggcaggtgcgggcaagtccttggttccgcgtggcagcgccggcgccggcgcgctcaatgatctgtgtattaaagtcaataactgggacctgttcttcagcccgagcgaggataactttaccaacgacttaaacaaaggcgaggagatcacgagcgatacgaacatcgaggcggcggaggaaaatattagcctggacctcattcagcagtactatctgacgttcaattttgacaatgagccggagaacatcagcattgaaaatctcagcagcgacatcatcggtcagttggaactgatgccgaacattgaacgctttccgaacggcaaaaaatatgaactggacaagtataccatgttccattacttacgcgcacaggaatttgagcacggcaagagccgcattgcgctgaccaatagcgttaacgaggccttgttaaatccgagccgtgtctacacgttcttcagcagcgattatgtcaaaaaagtgaacaaggcgaccgaagccgcgatgtttttgggctgggtcgagcaattggtttacgattttaccgacgaaaccagcgaggtgagcacgaccgacaaaattgcagatatcaccatcatcattccgtacatcggtccggcgctcaatatcggcaatatgttatacaaggacgactttgtgggcgcgctgatctttagcggcgcggttatcttattagaattcatcccggagatcgcaatcccggtcttgggcacctttgcgttggtgagctatatcgcgaataaagtgctcacggtccaaaccatcgataacgcgctcagcaagcgtaatgagaaatgggacgaggtttataagtatatcgtgaccaactggttagcaaaagtcaatacgcagatcgatctcatccgcaaaaaaatgaaagaagccttggaaaatcaagcggaggcaaccaaagccatcattaattaccagtataaccaatataccgaagaagaaaaaaacaatatcaacttcaatatcgatgatttgagcagcaaactgaacgagagcattaacaaagcgatgattaacatcaacaagttcttgaatcaatgcagcgtgagctatctcatgaacagcatgatcccgtatggcgtcaaacgcttggaagattttgacgccagcctgaaagatgcgctcctcaagtatatttatgacaaccgcggcaccctcattggccaggtggaccgcttgaaggataaagtgaacaatacgctcagcacggatatcccgttccagctgagcaagtacgtcgacaaccagcgcttactgagcacctttaccgagtatatcaagaacatcattaataccagcatcctcaacttgcgctatgagagcaatcacctgatcgacctcagccgctacgccagcaagatcaacatcggcagcaaggtcaatttcgacccgatcgataagaatcagatccaattgtttaacctggaaagcagcaagatcgaggttatcttgaagaacgcgattgtgtacaacagcatgtacgagaactttagcacgagcttctggattcgtatcccgaagtatttcaatagcattagcctgaataacgaatataccattatcaactgcatggaaaataatagcggctggaaggtgagcttaaattacggcgagatcatttggaccttacaggatacccaagaaatcaaacagcgcgtcgtctttaagtatagccagatgatcaacatcagcgattacatcaaccgctggatcttcgtgaccatcaccaataatcgcttgaataatagcaagatttacatcaatggtcgcttgattgatcaaaaaccgatcagcaatctcggtaatatccatgccagcaataacatcatgtttaagttagacggttgccgcgatacccaccgctatatctggatcaagtattttaacttatttgataaggaactcaacgaaaaggaaattaaagacttatatgacaatcagagcaatagcggcatcctgaaggatttctggggcgactacctgcagtacgataagccgtactatatgttgaacttgtatgacccgaacaaatatgtcgatgtgaacaatgtgggtattcgtggctatatgtacttaaagggcccgcgtggtagcgtgatgaccacgaatatttacttaaacagcagcttataccgcggcacgaagtttattatcaagaagtatgccagcggcaacaaggacaatatcgtccgcaacaacgaccgtgtgtatattaacgtggtggtgaagaataaagagtaccgcttggccacgaatgcgagccaggcgggcgtggaaaaaatcttgagcgcgttggagatcccggacgtcggcaacctcagccaggttgtggtgatgaagtctaaaaacgaccagggcatcacgaacaagtgcaaaatgaatttgcaagataacaacggcaacgacatcggctttattggttttcaccagttcaataacatcgccaaactcgtggccagcaattggtataaccgccaaattgaacgcagcagccgcacgctcggctgtagctgggagttcatcccggtggacgatggctggggcgagcgcccgctcgcggcgccggctgccccggcgcccgccgcccctgctgcgcctgcgccggcagcgccagccgcggcaccggcggctccggcgccggccgcgcccgccgcaccggccccagccgcgcctgcagctgcaccggcggcgccggcacctgcggcaccggcggcaccggcccctgcggcaccggccgccgcgccggctgcacctgctccggccgcgccggcggcgccagcgccggcagcgccggcagcagcaccggcggcaccggcgccagctgcaccggcggcgccagctcctgcggcgccggccggagatctggtgccacgcggttccgcgaattcgagctccgtcgacaagctttggagccacccgcagttcgaaaaataa

SEQ ID NO: 9 - DNA, 4641 bp, Artificial Sequence
  PAY100-rBoNTA-PAY100
atgggtagcagccatcatcatcaccatcatggtagcctggttccgcgtagctcttctgcgtacccggcggcgccggcgccggcgtacccggcggcgccggcgccgtacgcgccggcggcgtacccggcggcgccggcgccggcgtacccggcggcgccggcgccgtacgcgccggcggcgtacccggcggcgccggcgccggcgtacccggcggcgccggcgccgtacgcgccggcggcgtacccggcggcgccggcgccggcgtacccggcggcgccggcgccgtacgcgccggcggcgtacccggcggcgccggcgccggcgtacccggcggcgccggcgccgtacgcgccggcgccatttgtgaacaagcagtttaactataaggacccggtgaacggtgtggatatcgcgtatatcaaaatcccgaatgcgggccagatgcaaccagtcaaggcgttcaagattcataacaagatttgggttattccggaacgtgataccttcaccaatccggaagaaggcgatttaaatccgccgccagaagccaaacaagtgccggtgagctactatgatagcacgtatcttagcaccgataatgaaaaagacaattacctgaagggcgtgaccaagttgttcgagcgcatctacagtaccgacttaggccgcatgttgttgacgagcatcgttcgcggtatcccgttctggggcggctcgaccattgataccgagttgaaagtcattgacacgaactgtatcaatgttatccaaccggacggcagttatcgcagcgaggagttaaatttggtcatcatcggtccaagcgcagatattattcagttcgaatgcaagagcttcggccatgaggtcttgaatttgacgcgcaacggttacggcagcacccaatacatccgctttagcccggatttcacctttggcttcgaggagagcttggaggtggacaccaacccgctgttaggtgccggcaaattcgcaaccgacccggcagtgacgttggcgcacgaattgattcatgcgggtcaccgcttatacggtatcgcgatcaatccgaatcgcgtctttaaagtcaataccaacgcgtactacgaaatgagcggcttagaggttagctttgaagaattacgcaccttcggtggccacgacgccaagttcatcgacagcctgcaggaaaatgagttccgcttgtactattacaataaattcaaggacatcgcgagcaccttaaataaagcaaagagcattgtgggcaccaccgcaagcttgcagtacatgaagaacgtatttaaggaaaaatatttgttgtcggaggataccagcgggaaattcagcgtcgataagctgaaattcgacaaattgtataaaatgctgaccgagatttacaccgaggataacttcgtcaagttttttaaggtgttaaatcgtaagacctatttaaactttgataaagcggtgtttaaaattaatatcgtgccgaaggtgaattacaccatctacgatggtttcaatttacgcaacacgaatctggcggcgaattttaatggccaaaacaccgaaattaacaacatgaactttacgaagttaaagaatttcacgggcttattcgaattctacaagttattatgcgtgcgcggcatcattaccagcaaggcaggtgcgggcaagtccttggtaccgcgtggcagcgccggcgccggcgcgctcaatgatctgtgtattaaagtcaataactgggacctgttcttcagcccgagcgaggataactttaccaacgacttaaacaaaggcgaggagatcacgagcgatacgaacatcgaggcggcggaggaaaatattagcctggacctcattcagcagtactatctgacgttcaattttgacaatgagccggagaacatcagcattgaaaatctcagcagcgacatcatcggtcagttggaactgatgccgaacattgaacgctttccgaacggcaaaaaatatgaactggacaagtataccatgttccattacttacgcgcacaggaatttgagcacggcaagagccgcattgcgctgaccaatagcgttaacgaggccttgttaaatccgagccgtgtctacacgttcttcagcagcgattatgtcaaaaaagtgaacaaggcgaccgaagccgcgatgtttttgggctgggtcgagcaattggtttacgattttaccgacgaaaccagcgaggtgagcacgaccgacaaaattgcagatatcaccatcatcattccgtacatcggtccggcgctcaatatcggcaatatgttatacaaggacgactttgtgggcgcgctgatctttagcggcgcggttatcttattagaattcatcccggagatcgcaatcccggtcttgggcacctttgcgttggtgagctatatcgcgaataaagtgctcacggtccaaaccatcgataacgcgctcagcaagcgtaatgagaaatgggacgaggtttataagtatatcgtgaccaactggttagcaaaagtcaatacgcagatcgatctcatccgcaaaaaaatgaaagaagccttggaaaatcaagcggaggcaaccaaagccatcattaattaccagtataaccaatataccgaagaagaaaaaaacaatatcaacttcaatatcgatgatttgagcagcaaactgaacgagagcattaacaaagcgatgattaacatcaacaagttcttgaatcaatgcagcgtgagctatctcatgaacagcatgatcccgtatggcgtcaaacgcttggaagattttgacgccagcctgaaagatgcgctcctcaagtatatttatgacaaccgcggcaccctcattggccaggtggaccgcttgaaggataaagtgaacaatacgctcagcacggatatcccgttccagctgagcaagtacgtcgacaaccagcgcttactgagcacctttaccgagtatatcaagaacatcattaataccagcatcctcaacttgcgctatgagagcaatcacctgatcgacctcagccgctacgccagcaagatcaacatcggcagcaaggtcaatttcgacccgatcgataagaatcagatccaattgtttaacctggaaagcagcaagatcgaggttatcttgaagaacgcgattgtgtacaacagcatgtacgagaactttagcacgagcttctggattcgtatcccgaagtatttcaatagcattagcctgaataacgaatataccattatcaactgcatggaaaataatagcggctggaaggtgagcttaaattacggcgagatcatttggaccttacaggatacccaagaaatcaaacagcgcgtcgtctttaagtatagccagatgatcaacatcagcgattacatcaaccgctggatcttcgtgaccatcaccaataatcgcttgaataatagcaagatttacatcaatggtcgcttgattgatcaaaaaccgatcagcaatctcggtaatatccatgccagcaataacatcatgtttaagttagacggttgccgcgatacccaccgctatatctggatcaagtattttaacttatttgataaggaactcaacgaaaaggaaattaaagacttatatgacaatcagagcaatagcggcatcctgaaggatttctggggcgactacctgcagtacgataagccgtactatatgttgaacttgtatgacccgaacaaatatgtcgatgtgaacaatgtgggtattcgtggctatatgtacttaaagggcccgcgtggtagcgtgatgaccacgaatatttacttaaacagcagcttataccgcggcacgaagtttattatcaagaagtatgccagcggcaacaaggacaatatcgtccgcaacaacgaccgtgtgtatattaacgtggtggtgaagaataaagagtaccgcttggccacgaatgcgagccaggcgggcgtggaaaaaatcttgagcgcgttggagatcccggacgtcggcaacctcagccaggttgtggtgatgaagtctaaaaacgaccagggcatcacgaacaagtgcaaaatgaatttgcaagataacaacggcaacgacatcggctttattggttttcaccagttcaataacatcgccaaactcgtggccagcaattggtataaccgccaaattgaacgcagcagccgcacgctcggctgtagctgggagttcatcccggtggacgatggctggggcgagcgcccgctcgcggcgtacccggcggcgccggcgccggcgtacccggcggcgccggcgccgtacgcgccggcggcgtacccggcggcgccggcgccggcgtacccggcggcgccggcgccgtacgcgccggcggcgtacccggcggcgccggcgccggcgtacccggcggcgccggcgccgtacgcgccggcggcgtacccggcggcgccggcgccggcgtacccggcggcgccggcgccgtacgcgccggcggcgtacccggcggcgccggcgccggcgtacccggcggcgccggcgccgtacgcgccggcgggagatctggtgccacgcggttccgcgaattcgagctccgtcgacaagctttggagccacccgcagttcgaaaaataa

SEQ ID NO: 10 - DNA, 4641 bp, Artificial Sequence
  PAQ100-rBoNTA-PAQ100
atgggtagcagccatcatcatcaccatcatggtagcctggttccgcgtagctcttctgcgcagccggcggcgccggcgccggcgcagccggcggcgccggcgccgcaggcgccggcggcgcagccggcggcgccggcgccggcgcagccggcggcgccggcgccgcaggcgccggcggcgcagccggcggcgccggcgccggcgcagccggcggcgccggcgccgcaggcgccggcggcgcagccggcggcgccggcgccggcgcagccggcggcgccggcgccgcaggcgccggcggcgcagccggcggcgccggcgccggcgcagccggcggcgccggcgccgcaggcgccggcgccatttgtgaacaagcagtttaactataaggacccggtgaacggtgtggatatcgcgtatatcaaaatcccgaatgcgggccagatgcaaccagtcaaggcgttcaagattcataacaagatttgggttattccggaacgtgataccttcaccaatccggaagaaggcgatttaaatccgccgccagaagccaaacaagtgccggtgagctactatgatagcacgtatcttagcaccgataatgaaaaagacaattacctgaagggcgtgaccaagttgttcgagcgcatctacagtaccgacttaggccgcatgttgttgacgagcatcgttcgcggtatcccgttctggggcggctcgaccattgataccgagttgaaagtcattgacacgaactgtatcaatgttatccaaccggacggcagttatcgcagcgaggagttaaatttggtcatcatcggtccaagcgcagatattattcagttcgaatgcaagagcttcggccatgaggtcttgaatttgacgcgcaacggttacggcagcacccaatacatccgctttagcccggatttcacctttggcttcgaggagagcttggaggtggacaccaacccgctgttaggtgccggcaaattcgcaaccgacccggcagtgacgttggcgcacgaattgattcatgcgggtcaccgcttatacggtatcgcgatcaatccgaatcgcgtctttaaagtcaataccaacgcgtactacgaaatgagcggcttagaggttagctttgaagaattacgcaccttcggtggccacgacgccaagttcatcgacagcctgcaggaaaatgagttccgcttgtactattacaataaattcaaggacatcgcgagcaccttaaataaagcaaagagcattgtgggcaccaccgcaagcttgcagtacatgaagaacgtatttaaggaaaaatatttgttgtcggaggataccagcgggaaattcagcgtcgataagctgaaattcgacaaattgtataaaatgctgaccgagatttacaccgaggataacttcgtcaagttttttaaggtgttaaatcgtaagacctatttaaactttgataaagcggtgtttaaaattaatatcgtgccgaaggtgaattacaccatctacgatggtttcaatttacgcaacacgaatctggcggcgaattttaatggccaaaacaccgaaattaacaacatgaactttacgaagttaaagaatttcacgggcttattcgaattctacaagttattatgcgtgcgcggcatcattaccagcaaggcaggtgcgggcaagtccttggtaccgcgtggcagcgccggcgccggcgcgctcaatgatctgtgtattaaagtcaataactgggacctgttcttcagcccgagcgaggataactttaccaacgacttaaacaaaggcgaggagatcacgagcgatacgaacatcgaggcggcggaggaaaatattagcctggacctcattcagcagtactatctgacgttcaattttgacaatgagccggagaacatcagcattgaaaatctcagcagcgacatcatcggtcagttggaactgatgccgaacattgaacgctttccgaacggcaaaaaatatgaactggacaagtataccatgttccattacttacgcgcacaggaatttgagcacggcaagagccgcattgcgctgaccaatagcgttaacgaggccttgttaaatccgagccgtgtctacacgttcttcagcagcgattatgtcaaaaaagtgaacaaggcgaccgaagccgcgatgtttttgggctgggtcgagcaattggtttacgattttaccgacgaaaccagcgaggtgagcacgaccgacaaaattgcagatatcaccatcatcattccgtacatcggtccggcgctcaatatcggcaatatgttatacaaggacgactttgtgggcgcgctgatctttagcggcgcggttatcttattagaattcatcccggagatcgcaatcccggtcttgggcacctttgcgttggtgagctatatcgcgaataaagtgctcacggtccaaaccatcgataacgcgctcagcaagcgtaatgagaaatgggacgaggtttataagtatatcgtgaccaactggttagcaaaagtcaatacgcagatcgatctcatccgcaaaaaaatgaaagaagccttggaaaatcaagcggaggcaaccaaagccatcattaattaccagtataaccaatataccgaagaagaaaaaaacaatatcaacttcaatatcgatgatttgagcagcaaactgaacgagagcattaacaaagcgatgattaacatcaacaagttcttgaatcaatgcagcgtgagctatctcatgaacagcatgatcccgtatggcgtcaaacgcttggaagattttgacgccagcctgaaagatgcgctcctcaagtatatttatgacaaccgcggcaccctcattggccaggtggaccgcttgaaggataaagtgaacaatacgctcagcacggatatcccgttccagctgagcaagtacgtcgacaaccagcgcttactgagcacctttaccgagtatatcaagaacatcattaataccagcatcctcaacttgcgctatgagagcaatcacctgatcgacctcagccgctacgccagcaagatcaacatcggcagcaaggtcaatttcgacccgatcgataagaatcagatccaattgtttaacctggaaagcagcaagatcgaggttatcttgaagaacgcgattgtgtacaacagcatgtacgagaactttagcacgagcttctggattcgtatcccgaagtatttcaatagcattagcctgaataacgaatataccattatcaactgcatggaaaataatagcggctggaaggtgagcttaaattacggcgagatcatttggaccttacaggatacccaagaaatcaaacagcgcgtcgtctttaagtatagccagatgatcaacatcagcgattacatcaaccgctggatcttcgtgaccatcaccaataatcgcttgaataatagcaagatttacatcaatggtcgcttgattgatcaaaaaccgatcagcaatctcggtaatatccatgccagcaataacatcatgtttaagttagacggttgccgcgatacccaccgctatatctggatcaagtattttaacttatttgataaggaactcaacgaaaaggaaattaaagacttatatgacaatcagagcaatagcggcatcctgaaggatttctggggcgactacctgcagtacgataagccgtactatatgttgaacttgtatgacccgaacaaatatgtcgatgtgaacaatgtgggtattcgtggctatatgtacttaaagggcccgcgtggtagcgtgatgaccacgaatatttacttaaacagcagcttataccgcggcacgaagtttattatcaagaagtatgccagcggcaacaaggacaatatcgtccgcaacaacgaccgtgtgtatattaacgtggtggtgaagaataaagagtaccgcttggccacgaatgcgagccaggcgggcgtggaaaaaatcttgagcgcgttggagatcccggacgtcggcaacctcagccaggttgtggtgatgaagtctaaaaacgaccagggcatcacgaacaagtgcaaaatgaatttgcaagataacaacggcaacgacatcggctttattggttttcaccagttcaataacatcgccaaactcgtggccagcaattggtataaccgccaaattgaacgcagcagccgcacgctcggctgtagctgggagttcatcccggtggacgatggctggggcgagcgcccgctcgcggcgcagccggcggcgccggcgccggcgcagccggcggcgccggcgccgcaggcgccggcggcgcagccggcggcgccggcgccggcgcagccggcggcgccggcgccgcaggcgccggcggcgcagccggcggcgccggcgccggcgcagccggcggcgccggcgccgcaggcgccggcggcgcagccggcggcgccggcgccggcgcagccggcggcgccggcgccgcaggcgccggcggcgcagccggcggcgccggcgccggcgcagccggcggcgccggcgccgcaggcgccggcgggagatctggtgccacgcggttccgcgaattcgagctccgtcgacaagctttggagccacccgcagttcgaaaaataa

SEQ ID NO: 11 - DNA, 4641 bp, Artificial Sequence
  PAT100-rBoNTA-PAT100
atgggtagcagccatcatcatcaccatcatggtagcctggttccgcgtagctcttctgcgaccccggcggcgccggcgccggcgaccccggcggcgccggcgccgaccgcgccggcggcgaccccggcggcgccggcgccggcgaccccggcggcgccggcgccgaccgcgccggcggcgaccccggcggcgccggcgccggcgaccccggcggcgccggcgccgaccgcgccggcggcgaccccggcggcgccggcgccggcgaccccggcggcgccggcgccgaccgcgccggcggcgaccccggcggcgccggcgccggcgaccccggcggcgccggcgccgaccgcgccggcgccatttgtgaacaagcagtttaactataaggacccggtgaacggtgtggatatcgcgtatatcaaaatcccgaatgcgggccagatgcaaccagtcaaggcgttcaagattcataacaagatttgggttattccggaacgtgataccttcaccaatccggaagaaggcgatttaaatccgccgccagaagccaaacaagtgccggtgagctactatgatagcacgtatcttagcaccgataatgaaaaagacaattacctgaagggcgtgaccaagttgttcgagcgcatctacagtaccgacttaggccgcatgttgttgacgagcatcgttcgcggtatcccgttctggggcggctcgaccattgataccgagttgaaagtcattgacacgaactgtatcaatgttatccaaccggacggcagttatcgcagcgaggagttaaatttggtcatcatcggtccaagcgcagatattattcagttcgaatgcaagagcttcggccatgaggtcttgaatttgacgcgcaacggttacggcagcacccaatacatccgctttagcccggatttcacctttggcttcgaggagagcttggaggtggacaccaacccgctgttaggtgccggcaaattcgcaaccgacccggcagtgacgttggcgcacgaattgattcatgcgggtcaccgcttatacggtatcgcgatcaatccgaatcgcgtctttaaagtcaataccaacgcgtactacgaaatgagcggcttagaggttagctttgaagaattacgcaccttcggtggccacgacgccaagttcatcgacagcctgcaggaaaatgagttccgcttgtactattacaataaattcaaggacatcgcgagcaccttaaataaagcaaagagcattgtgggcaccaccgcaagcttgcagtacatgaagaacgtatttaaggaaaaatatttgttgtcggaggataccagcgggaaattcagcgtcgataagctgaaattcgacaaattgtataaaatgctgaccgagatttacaccgaggataacttcgtcaagttttttaaggtgttaaatcgtaagacctatttaaactttgataaagcggtgtttaaaattaatatcgtgccgaaggtgaattacaccatctacgatggtttcaatttacgcaacacgaatctggcggcgaattttaatggccaaaacaccgaaattaacaacatgaactttacgaagttaaagaatttcacgggcttattcgaattctacaagttattatgcgtgcgcggcatcattaccagcaaggcaggtgcgggcaagtccttggtaccgcgtggcagcgccggcgccggcgcgctcaatgatctgtgtattaaagtcaataactgggacctgttcttcagcccgagcgaggataactttaccaacgacttaaacaaaggcgaggagatcacgagcgatacgaacatcgaggcggcggaggaaaatattagcctggacctcattcagcagtactatctgacgttcaattttgacaatgagccggagaacatcagcattgaaaatctcagcagcgacatcatcggtcagttggaactgatgccgaacattgaacgctttccgaacggcaaaaaatatgaactggacaagtataccatgttccattacttacgcgcacaggaatttgagcacggcaagagccgcattgcgctgaccaatagcgttaacgaggccttgttaaatccgagccgtgtctacacgttcttcagcagcgattatgtcaaaaaagtgaacaaggcgaccgaagccgcgatgtttttgggctgggtcgagcaattggtttacgattttaccgacgaaaccagcgaggtgagcacgaccgacaaaattgcagatatcaccatcatcattccgtacatcggtccggcgctcaatatcggcaatatgttatacaaggacgactttgtgggcgcgctgatctttagcggcgcggttatcttattagaattcatcccggagatcgcaatcccggtcttgggcacctttgcgttggtgagctatatcgcgaataaagtgctcacggtccaaaccatcgataacgcgctcagcaagcgtaatgagaaatgggacgaggtttataagtatatcgtgaccaactggttagcaaaagtcaatacgcagatcgatctcatccgcaaaaaaatgaaagaagccttggaaaatcaagcggaggcaaccaaagccatcattaattaccagtataaccaatataccgaagaagaaaaaaacaatatcaacttcaatatcgatgatttgagcagcaaactgaacgagagcattaacaaagcgatgattaacatcaacaagttcttgaatcaatgcagcgtgagctatctcatgaacagcatgatcccgtatggcgtcaaacgcttggaagattttgacgccagcctgaaagatgcgctcctcaagtatatttatgacaaccgcggcaccctcattggccaggtggaccgcttgaaggataaagtgaacaatacgctcagcacggatatcccgttccagctgagcaagtacgtcgacaaccagcgcttactgagcacctttaccgagtatatcaagaacatcattaataccagcatcctcaacttgcgctatgagagcaatcacctgatcgacctcagccgctacgccagcaagatcaacatcggcagcaaggtcaatttcgacccgatcgataagaatcagatccaattgtttaacctggaaagcagcaagatcgaggttatcttgaagaacgcgattgtgtacaacagcatgtacgagaactttagcacgagcttctggattcgtatcccgaagtatttcaatagcattagcctgaataacgaatataccattatcaactgcatggaaaataatagcggctggaaggtgagcttaaattacggcgagatcatttggaccttacaggatacccaagaaatcaaacagcgcgtcgtctttaagtatagccagatgatcaacatcagcgattacatcaaccgctggatcttcgtgaccatcaccaataatcgcttgaataatagcaagatttacatcaatggtcgcttgattgatcaaaaaccgatcagcaatctcggtaatatccatgccagcaataacatcatgtttaagttagacggttgccgcgatacccaccgctatatctggatcaagtattttaacttatttgataaggaactcaacgaaaaggaaattaaagacttatatgacaatcagagcaatagcggcatcctgaaggatttctggggcgactacctgcagtacgataagccgtactatatgttgaacttgtatgacccgaacaaatatgtcgatgtgaacaatgtgggtattcgtggctatatgtacttaaagggcccgcgtggtagcgtgatgaccacgaatatttacttaaacagcagcttataccgcggcacgaagtttattatcaagaagtatgccagcggcaacaaggacaatatcgtccgcaacaacgaccgtgtgtatattaacgtggtggtgaagaataaagagtaccgcttggccacgaatgcgagccaggcgggcgtggaaaaaatcttgagcgcgttggagatcccggacgtcggcaacctcagccaggttgtggtgatgaagtctaaaaacgaccagggcatcacgaacaagtgcaaaatgaatttgcaagataacaacggcaacgacatcggctttattggttttcaccagttcaataacatcgccaaactcgtggccagcaattggtataaccgccaaattgaacgcagcagccgcacgctcggctgtagctgggagttcatcccggtggacgatggctggggcgagcgcccgctcgcggcgaccccggcggcgccggcgccggcgaccccggcggcgccggcgccgaccgcgccggcggcgaccccggcggcgccggcgccggcgaccccggcggcgccggcgccgaccgcgccggcggcgaccccggcggcgccggcgccggcgaccccggcggcgccggcgccgaccgcgccggcggcgaccccggcggcgccggcgccggcgaccccggcggcgccggcgccgaccgcgccggcggcgaccccggcggcgccggcgccggcgaccccggcggcgccggcgccgaccgcgccggcgggagatctggtgccacgcggttccgcgaattcgagctccgtcgacaagctttggagccacccgcagttcgaaaaataa